(19) United States
(12) Patent Application Publication
(10) Pub. No.: US 2026/0345524 A1
(43) Pub. Date: Nov. 5, 2026

(54) TARGETING KDM2A TO ENHANCE IMMUNE RESPONSIVENESS TO CANCERS

(71) Applicant: The Board of Trustees of the Leland Stanford Junior University, Stanford, CA (US)

(72) Inventors: John B. Sunwoo, Redwood City, CA (US); June H. Shin, Redwood City, CA (US); Chen Chen, Redwood City, CA (US)

(21) Appl. No.: 19/487,353

(22) PCT Filed: May 30, 2024
(86) PCT No.: PCT/US2024/031758

§ 371 Date: Nov. 24, 2025

Related U.S. Application Data
(60) Provisional application No. 63/470,654, filed on Jun. 2, 2023.

(51) Int. Cl. A61K 31/15; A61K 40/11; A61K 40/31; A61K 40/42; A61P 35/00

(52) U.S. Cl. CPC A61K 31/15; A61K 40/11; A61K 40/31; A61K 40/4211; A61P 35/00; A61K 2239/13

(57) ABSTRACT

Tumors comprising cancer cells with decreased expression of NSD1 can have an immune-cold tumor microenvironment (TME), where within the TME, T cells capable of acting against the cancer cells are reduced in number and are resistant to T cell infiltration. Expression of chemokines involved in T cell recruitment, e.g. CXCL9 and CXCL10, is shown to be repressed in the context of NSD1 inactivation. Inhibition of KDM2A induces the increased methylation of H3K36 and decreased methylation of H3K27, resulting in both the increased expression of CXCL9/CXCL10 and the increased infiltration of T cells into the TME. KDM2A inhibition can enhance clinical response to immune checkpoint inhibitors.

### CROSS REFERENCE TO OTHER APPLICATIONS

This application is a U.S. National Stage of PCT Application No. PCT/US2024/031758, filed May 30, 2024, which claims the benefit of U.S. Provisional Application No. 63/470,654, filed Jun. 2, 2023, the contents of which are hereby incorporated by reference in their entirety.

### GOVERNMENT SUPPORT RESEARCH

This invention was made with Government support under contract DE030054 awarded by the National Institutes of Health. The Government has certain rights in the invention.

### BACKGROUND

Head and neck squamous cell carcinoma (HNSCC), the 6th most common malignancy worldwide, is a devastating malignancy of the mucosal lining of the upper aerodigestive tract. The aggressive nature of the cancer, as well as the treatment itself, have significant impact on critical functions like speech, breathing, and swallowing. Despite improved multi-modal treatment strategies, patients with advanced HNSCC still suffer from a low survival rate.

Recently, immunotherapy approaches with checkpoint blocking antibodies, such as pembrolizumab and nivolumab, have shown impressive responses in recurrent/metastatic HNSCC, though only in a minority of patients. In general, the overall response rate to immune checkpoint blockade is limited, and in HNSCC, it is <20%. Numerous strategies to enhance endogenous and synthetic immune-mediated rejection of tumors are under intense investigation; however, all face significant challenges pending better understanding of the interface between tumor cells and the immune system within the tumor microenvironment. A particular challenge for immune checkpoint blockade approaches (and all immunotherapy approaches for cancer) has been the development of ways to overcome tumor microenvironments that lack (or exclude/prevent) immune cell infiltration; what is sometimes called an immune-cold phenotype.

Previous analyses of multiomics data from The Cancer Genome Atlas (TCGA) revealed a distinct immune-cold subset of HNSCC tumors with a particularly low level of tumor associated T cells. This human papilloma virus (HPV)-negative subset of HNSCC was also unique in that 57% of these tumors had inactivating mutations in the histone methyl transferase NSD1 (nuclear receptor binding SET domain protein 1). Further, all HNSCC tumors in the TCGA with NSD1 inactivating mutations have multiomics profiles placing them in the immune-cold subset.

Methods of increasing immune cell (e.g. T cell) presence in immune-cold tumors is of great interest for furthering the treatment of these cancers. The present disclosure addresses this issue.

### SUMMARY

Compositions and methods are provided for enhancing the immune response to tumors, e.g. solid tumors, lymphomas, etc. In some embodiments, immune responsiveness is enhanced by inhibiting activity of lysine-specific demethylase 2A (KDM2A) in the tumor cells. In some embodiments the solid tumors comprise cancer cells deficient in histone methyl transferase NSD1 (nuclear receptor binding SET domain protein 1). In some embodiments the cancer cells comprise mutations that inactivate or decrease expression of NSD1. In some embodiments an inactivating mutation is present in one or both copies of the NSD1 gene. In some embodiments an individual is phenotyped or genotyped for NSD1 prior to treatment, where an individual with an inactivating mutation in NSD1; or reduced activity of NSD1, is selected for treatment.

It is shown herein that tumors comprising cancer cells with decreased expression of NSD1 can have an immune-cold tumor microenvironment (TME), where within the TME, T cells capable of acting against the cancer cells are reduced in number and are resistant to T cell infiltration. For example, expression of chemokines involved in T cell recruitment, e.g. CXCL9 and CXCL10, is shown to be repressed in the context of NSD1 inactivation. Inhibition of KDM2A induces the increased methylation of H3K36 and decreased methylation of H3K27, resulting in both the increased expression of CXCL9/CXCL10 and the increased infiltration of T cells into the TME. Since response rates to immune checkpoint inhibitors (ICI) correlate with the extent of immune cell infiltration in the TME, KDM2A inhibition can enhance clinical response to ICI.

In some embodiments an individual selected for treatment has a carcinoma. In some embodiments the carcinoma is a squamous cell carcinoma. In some embodiments the squamous cell carcinoma is a head and neck squamous cell carcinoma (HNSCC).

In some embodiments an individual selected for treatment is administered an effective dose of an inhibitor of KDM2A. In some embodiments, the individual is treated for a solid tumor comprising cancer cells deficient in histone methyl transferase NSD1 (nuclear receptor binding SET domain protein 1). In some embodiments the inhibitor is daminozide. In some embodiments, administration of a KDM2A inhibitor is combined with administration of an effective dose of an immune checkpoint inhibitor (ICI), including, without limitation, inhibitors of cytotoxic T-lymphocyte-associated antigen 4 (CTLA4; also known as CD152); programmed cell death protein 1 (PD1; also known as CD279); and PD1 ligand, PDL1.

In some embodiments, an inhibitor of KDM2A is administered for treatment of cancer in combination with adoptive cell therapies to enhance effector cell infiltration into the tumor microenvironment. In some embodiments, the individual is treated for a solid tumor comprising cancer cells deficient in histone methyl transferase NSD1 (nuclear receptor binding SET domain protein 1). In some embodiments the adoptive cells are T cells, NKT cells, NK cells, macrophages, B cells, or other effector immune cells. In some embodiments the T cells are CAR T-cells or CAR-NK cells that recognize a tumor antigen present in the patient cancer. CAR T-cells are optionally patient derived.

In some embodiments an individual selected for treatment is administered an effective dose of an inhibitor of KDM2A, where the individual is treated for a solid tumor comprising cancer cells having a wild-type NSD1, where the KDM2A inhibitor is provided in combination with an immunotherapy, e.g. an immune checkpoint inhibitor, adoptive cell therapy, and the like.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIGS. 1A-1D: NSD1 inactivation downregulates the expression of CXCL9 and CXCL10 in HNSCC. RNA expression and genomic sequencing data from a multicenter, phase II, window-of-opportunity trial (NCT02296684) (23) was examined for CXCL9 and CXCL10 expression and NSD1 mutations. In this trial, neoadjuvant pembrolizumab was administered to patients with locally advanced, resectable, HPV-unrelated HNSCC two to three weeks prior to definitive surgical resection. (A) Comparison of CXCL9 and CXCL10 baseline expression to pathologic tumor response (pTR), as defined in (23), to the anti-PD-1 antibody pembrolizumab. Circles represent responders. ▴ A represent non-responders with wild-type NSD1. Gray triangles represent non-responders with mutated NSD1. (B) Tumor histology from the two patients in the trial that had tumors with NSD1 mutations. Histology of NSD wild-type tumors is also shown. Red=CD3 staining of T cells. Yellow=pancytokeratin staining of the tumor cells. Blue=DAPI. White scale bars represent ~100 μm. (C) Comparison of the CXCL9 and CXCL10 expression in NSD1 wild-type and mutated HPV-negative HNSCC tumors in the Cancer Genome Atlas. ****p<0.0001. (D) Expression of CXCL9 and CXCL10 in malignant cells of NSD1 wild-type and mutated primary and metastatic HNSCCs. Box plots show expression (Normalized counts) of CXCL9 and CXCL10 in malignant single cells (points) of HNSCC samples, including primary and metastatic (lymph node metastasis) tumors. Black and red boxes and points represent NSD1 wild-type and NSD1 mutated HNSCCs, respectively. Single cell RNA-Sequencing data was accessed from a previous study by Puram et al.

FIGS. 2A-2L: KDM2A inhibition restores CXCL9 and CXCL10 expression in the absence of NSD1. Quantitative RT-PCR (qRT-PCR) analysis of NSD1 expression after inhibition of NSD1 expression by shRNA transduction in human (FaDu) (A) and mouse (MOC1) (B) HNSCC cell lines. Representative Western blots of H3K36me2 and H3K27me3 levels in FaDu (C) and MOC1 (D) cells after inhibition of NSD1 expression by shRNA transduction. Quantification of mRNA expression by qRT-PCR of CXCL9 and CXCL10 in FaDu (E) and MOC1 (F) cells transduced to express NSD1 shRNA. Data shown are representative of experiments repeated at least three times. Error bars represent standard error of the mean (SEM), **p<0.01, ***p≤50.001. (G) Reciprocal relationship of H3K36me2 and H3K27me3. NSD1 catalyzes the di-methylation of H3K36 (H3K36me2). KDM2A is a lysine demethylase with specificity for H3K36me2. Tri-methylation of H3K27 (H3K27me3) is directly antagonized by H3K36me2 (and H3K36me3). (H) KDM2A mRNA expression (assessed by qRT-PCR and normalized to HPRT1) in FaDu and MOC1 cell lines, transduced to express shRNA targeting NSD1 with and without shRNA targeting KDM2A. H3K36me2 and H3K27me3 levels were assessed by Western blot analysis of FaDu (I) and MOC1 (J) cells, transduced to express shRNA targeting NSD1 with and without shRNA targeting KDM2A. All experiments were repeated at least three times. Error bars represent standard error of the mean (SEM), *p<0.05, **p<0.01, ***p≤50.001. CXCL9 and CXCL10 mRNA expression levels were assessed by qRT-PCR and normalized to expression of HPRT1 in FaDu (K) and MOC1 (L) cell lines, transduced to express shRNA targeting NSD1 with and without shRNA targeting KDM2A. Data shown are representative of experiments repeated at least three times. Error bars represent standard error of the mean (SEM), *p<0.05, ***p≤50.001.

FIG. 3A-3G. KDM2A inhibition reverses the immune cold phenotype induced by NSD1 inactivation and induces T cell infiltration into the tumor microenvironment. (A) Overview and illustration of experiment workflow. Tumor spheroids were established from HNSCC tumor cells transfected to express truncated CD19 on the cell surface and grown in submerged Matrigel. The spheroids were co-cultured with CD19-CAR-T cells for three days and then isolated for analysis by confocal microscopy. (B) Assessment by qRT-PCR of CXCL9 and CXCL10 expression after CXCL9 or CXCL10 overexpression (OE) in FaDu cells transduced to express shRNA targeting NSD1 (NSD1-sh). (C) Representative images of tumor spheroids after co-cultured with CD19-CAR-T cells for 3 days. Ctrl=vector control; NSD1-sh=shRNA targeting NSD1; CXCL9-OE=overexpression construct for CXCL9; CXCL10-OE=overexpression construct for CXCL10; KDM2A-sh=shRNA targeting KDM2A. Scale bar=50 um. (D) Representative images of tumor spheroids stained with anti-CD3 antibody (red) and DAPI (blue). Scale bar=50 um. (E) Quantification of CD3+ cells per 100 cells in the spheroids. (F) Representative images of FaDu tumor spheroids expressing control or NSD1 targeted shRNA with or without the KDM2A inhibitor (daminozide) treatment. The spheroids were stained with anti-CD3 antibody (red) and DAPI (blue). Scale bar=50 um. (G) Quantification of CD3⁺ cells per 100 cells in the spheroids. Data shown are representative of experiments repeated at least three times. Error bars represent standard error of the mean (SEM). *p<0.05, **p<0.01, ***p≤50.001.

FIGS. 4A-4F. KDM2A inhibition induces T cell infiltration of the tumor microenvironment in vivo. Growth curves of MOC1 control (vector control) and NSD1 knockdown (NSD1-sh) tumors in syngeneic Rag1^{-/-} (Rag1 KO) mice (n=6 for each group) (A) and wild-type mice (n=8 per group) (B). (C) Tumor infiltrating T cells (CD45⁺CD3⁺) were quantified by flow cytometry of dissociated tumors. Growth curves of MOC1 control, NSD1 knockdown (NSD1-sh) and NSD1 and KDM2A double-knockdown (NSD1-sh & KDM2A-sh) cells in Rag1 KO mice (n=6 for each group) (D) and wild-type mice (n=5 for each group) (E). (F) Representative immunofluorescence images of CD3, CXCL9, and CXCL10 expression (red) in tumors formed from MOC1 cells transduced with vector control (Ctrl), NSD1 shRNA (NSD1-sh), or both NSD1 shRNA and KDM2A shRNA (NSD1-sh&KDM2A-sh). Scale bar=50 um. Blue stain=DAPI. Error bars represent standard error of the mean (SEM). ***p≤50.001. ns=not significant.

FIG. 5. Statistical analysis of NSD1 mutation data and CXCL9/CXCL10 expression data presented in FIG. 1. Here, RNA expression and genomic sequencing data from a multicenter, phase II, window-of-opportunity pembrolizumab trial (NCT02296684) was examined for CXCL9 and CXCL10 expression and NSD1 mutations. Comparison of CXCL9 and CXCL10 expression to pathologic tumor response to the anti-PD-1 antibody pembrolizumab (see FIG. 1). Circles represent responders. ▴ represent non-responders with wild-type NSD1. Gray triangles represent non-responders with mutated NSD1. For statistical comparisons, unpaired t-test was used. Error bars represent standard error of the mean (SEM), **p<0.01, ***p≤0.001.

FIG. 6. Expression of T cell related cytokine genes in head and neck squamous cell carcinoma tumors. Data from the HPV-negative HNSCC samples in the Cancer Genome Atlas (TCGA) database was analyzed using cBioportal. The T cell related cytokine genes with the greatest differences in expression between tumors with NSD1 mutations and those with wild-type NSD1. *p<0.05, **p<0.01, ***p≤0.001, ****p≤0.0001.

FIG. 7. ChIP-seq data was accessed from a previous study published by Nargess et al. (1) Integrated genome viewer visualization of CXCL9 and CXCL10 ChIP-seq peaks for FaDu, Cal-27 and Detroit-562 control and NSD1 knockout cell lines.

FIG. 8. Expression of H3K27me3 related genes in head and neck squamous cell carcinoma tumors. Data from the HPV-negative HNSCC samples in the Cancer Genome Atlas (TCGA) database was analyzed using cBioportal. The H3K27me3 related genes with the greatest differences in expression between tumors with NSD1 mutations and those with wild-type NSD1. *p<0.05, **p<0.01, ***p≤0.001, ****p≤0.0001.

FIGS. 9A-9D. Expression of lysine demethylases (KDM) in head and neck squamous cell carcinoma. (A) KDM mRNA expression among HPV-negative HNSCC tumor samples in TCGA database. (B) KDM mRNA expression in FaDu cells using published RNA sequencing data (2). (C) KDM2A, KDM4A, KDM4B and KDM4C mRNA expression in FaDu, assessed by qRT-PCR, using delta Ct, and normalized to HPRT1. (D) KDM2A, KDM4A, KDM4B and KDM4C mRNA expression, assessed by qRT-PCR, using delta Ct, and normalized to HPRT1, in FaDu cells transduced to express shRNA targeting NSD1 with or without shRNA targeting KDM2A. Error bars represent standard error of the mean (SEM). *p<0.05, ***p≤0.001.

FIG. 10. CXCL9 and CXCL10 protein expression in FaDu cell line, tranduced to express shRNA for NSD1 and/or KDM2A or expression constructs for CXCL9 and CXCL10. Protein expression was assessed in the conditioned medium by ELISA. Error bars represent standard error of the mean (SEM). *p<0.05, ***p≤0.001.

FIG. 11. No difference in growth kinetics of HNSCC cells in the presence of NSD1 and KDM2A inhibition. Cell growth was measured by xCelligence impedance assay. Cell index plots for MOC1 vector control (Ctrl), NSD1 knockdown (NSD1-sh) and NSD1 and KDM2A double knockdown (NSD1-sh&KDM2A-sh) are shown.

FIG. 12. Knockout of NSD1 in MOC1 cells using CRISPR/Cas9. (A) Quantification of the NSD1 deletion editing by PCR. Shown is NSD1 edited area expression relative to un-edited area expression (3) (B) Quantitative RT-PCR analysis of KDM2A expression after KDM2A shRNA knockdown. (C) Assessment of H3K36me2 and H3K27me3 expression levels by Western blot in MOC1 cells after NSD1 knockout with or without KDM2A knockdown. (D-E) Tumor growth curves of MOC1 control, NSD1 knockout (NSD1-KO) and NSD1-KO with KDM2A shRNA knockdown (NSD1-KO & KDM2A-sh) cells in Rag1 KO mice (n=6 for each group) (D) and C57BL/6 wild-type mice (n=6 for each group) (E).

FIG. 13. (A-B) Quantification of CXCL9 and CXCL10 protein expression in tumors from the mouse experiments in FIG. 4F, measured by immunofluorescence antibody staining as Integrated Density per Area. Error bars represent standard error of the mean (SEM). **p<0.01, ***p≤0.001, ****p≤0.0001. (C-D) CXCL9 and CXCL10 mRNA expression, relative to Hprt, was measured by qRT-PCR (using delta Ct), using RNA extracted from the mouse tumors formed from MOC1 cells transduced with vector control (Ctrl), NSD1 shRNA (NSD1-sh), or both NSD1 shRNA and KDM2A shRNA (NSD1-sh&KDM2A-sh) in FIG. 4F.

FIG. 14. Luciferase expressing MOC1 cells, transduced with scramble control shRNA, NSD1 shRNA, or both NSD1 shRNA and KDM2A shRNA, were injected to B6 wild type mice subcutaneously. The mice were treated 24 hours later with or without EZH2 inhibitor (EPZ-6438) at a dose of 200 mg/kg daily, with or without anti-PD-1 antibody (200 μg/mouse). Bioluminescence imaging was used to assess tumor burden; quantified luminescence signal on day 10 is shown in photons per second. *p<0.05.

### DETAILED DESCRIPTION

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As used herein, compounds which are “commercially available” may be obtained from commercial sources including but not limited to Acros Organics (Pittsburgh PA), Aldrich Chemical (Milwaukee WI, including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester PA), Crescent Chemical Co. (Hauppauge NY), Eastman Organic Chemicals, Eastman Kodak Company (Rochester NY), Fisher Scientific Co. (Pittsburgh PA), Fisons Chemicals (Leicestershire UK), Frontier Scientific (Logan UT), ICN Biomedicals, Inc. (Costa Mesa CA), Key Organics (Cornwall U.K.), Lancaster Synthesis (Windham NH), Maybridge Chemical Co. Ltd. (Cornwall U.K.), Parish Chemical Co. (Orem UT), Pfaltz & Bauer, Inc. (Waterbury CN), Polyorganix (Houston TX), Pierce Chemical Co. (Rockford IL), Riedel de Haen AG (Hannover, Germany), Spectrum Quality Product, Inc. (New Brunswick, NJ), TCI America (Portland OR), Trans World Chemicals, Inc. (Rockville MD), Wako Chemicals USA, Inc. (Richmond VA), Novabiochem and Argonaut Technology.

Compounds can also be made by methods known to one of ordinary skill in the art. As used herein, “methods known to one of ordinary skill in the art” may be identified though various reference books and databases. Suitable reference books and treatises that detail the synthesis of reactants useful in the preparation of compounds of the present invention, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services.

NSD1 is a SET-domain containing histone methyltransferase that catalyzes di-methylation of histone 3 specifically at lysine 36 (H3K36me2). Synthesis of the key chromatin silencing modification tri-methylation of H3K27 (H3K27me3) by the Polycomb repressive complex (PRC2) is directly antagonized by H3K36 di- and tri-methylation.

A change involving the NSD1 gene is associated with acute myeloid leukemia. This change occurs when part of chromosome 5 breaks off and reattaches to part of chromosome 11. The translocation abnormally fuses the NSD1 gene on chromosome 5 with the NUP98 gene on chromosome 11. The fused NUP98-NSD1 gene turns on genes that promote the growth of immature blood cells and blocks processes that would turn the genes off. A different type of alteration involving the NSD1 gene is associated with neuroblastoma and glioma, where promoter hypermethylation, turns off the production of the NSD1 enzyme. A number of somatic mutations that result in a truncated and inactive NSD1 gene have been reported in cancer patients.

Cancer cells may be genotyped to determine the presence of NSD1 mutations, using methods known in the art. For example, the genotyping may comprise collecting a cancer cell sample; extracting DNA; targeting the NSD1 gene by polymerase chain reaction (PCR), DNA sequencing, or microarray analysis to identify and analyze the genetic variations. The refseq summary for the genetic sequence may be accessed at Genbank, NM_022455.

Lysine-specific demethylase 2A (KDM2A) also known as F-box and leucine-rich repeat protein 11 (FBXL11) is a member of the superfamily of alpha-ketoglutarate-dependent hydroxylases, which are non-haem iron-containing proteins. The F-box protein family is characterized by an approximately 40 amino acid motif, the F-box. The F-box proteins constitute one of the four subunits of ubiquitin protein ligase complex called SCFs (SKP1-cullin-F-box), which function in phosphorylation-dependent ubiquitination. In addition to an F-box, it contains at least 6 highly degenerated leucine-rich repeats. FBXL11/KDM2A is a histone H3 lysine 36 demethylase enzyme. The enzymatic activity of FBXL11/KDM2A relies on a conserved JmjC domain in the N-terminus of the protein that co-ordinates iron and alphaketoglutarate to catalyze demethylation via a hydroxylation based mechanism. It has recently been demonstrated that a ZF-CxxC DNA binding domain within FBXL11/KDM2A has the capacity to interact with non-methylated DNA and this domain targets FBXL11/KDM2A to CpG island regions of the genome where it specifically removes histone H3 lysine 36 methylation. The refseq summary for the genetic sequence may be accessed at Genbank, NM_001256405.

Inhibitors, as used herein, refer to agents that reduce the expression or activity of KDM2A and include small molecules, anti-sense or RNAi agents, peptides, polypeptides, proteins, including more specifically antibodies, and a variety of others. An inhibitor may, for example, reduce the activity of KDM2A in a targeted cell by at least about 5-fold, 10-fold, 50-fold, 100-fold, 500-fold, or more. Inhibitors may be selective of KDM2A, or may be pan-KDM inhibitors.

Inhibitors of KDM2A known in the art include, without limitation:

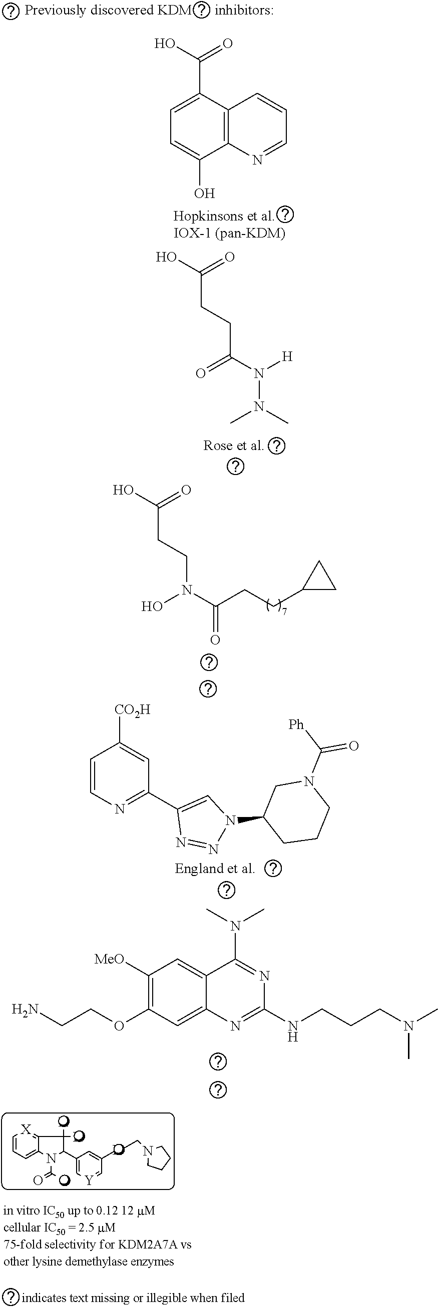

See, for example, Gerken et al. Angew Chem Int Ed Engl. 2017 Dec. 4; 56(49): 15555-15559, herein specifically incorporated by reference.

As an inhibitor of KDM2A, plant growth regulator Daminozide,

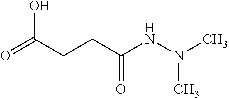

has been reported to significantly abrogate the effect of KDM2A on histone demethylation. KDM2A/7A-IN-1 is a first-in-class, selective and cell-permeable inhibitor of histone lysine demethylases KDM2A/7A, with an IC50 of 0.16 μM for KDM2A, exhibits 75 fold selectivity over other JmjC lysine demethylases, and is inactive on methyl transferases, and histone acetyl transferases.

The effective dose of an KDM2A inhibitor may range up to about 0.01 mg/kg, 0.05 mg/kg, 0.1 mg/kg, 0.5 mg/kg up to about 20 mg/kg, up to about 10 mg/kg, up to about 5 mg/kg; up to about 1 mg/kg, up to about 0.5 mg/kg; up to about 0.1 mg/kg; up to about 0.05 mg/kg; where the dose may vary with the specific agent and recipient.

As used herein, the terms “chimeric antigen receptor T-cell” and “CAR-Treg cell” are used interchangeably to refer to a T-cell that has been recombinantly modified to express a CAR. As used herein, the terms “chimeric antigen receptor” and “CAR” are used interchangeably to refer to a polyprotein comprising multiple functional domains arranged from amino to carboxy terminus in the sequence: (a) an antigen binding domain (ABD), (b) a transmembrane domain (TD); (c) one or more cytoplasmic signaling domains (CSDs) wherein the foregoing domains (a)-(c) may optionally be linked by one or more spacer domains. The CAR may also further comprise a signal peptide sequence which is conventionally removed during post-translational processing and presentation of the CAR on the cell surface. CARs useful in the practice of the present invention are prepared in accordance with principles well known in the art. See e.g., Eshhaar et al. U.S. Pat. No. 7,741,465 B1 issued Jun. 22, 2010; Sadelain, et al (2013) Cancer Discovery 3(4):388-398; Jensen and Riddell (2015) Current Opinions in Immunology 33:9-15; Gross, et al. (1989) PNAS(USA) 86(24):10024-10028; Curran, et al. (2012) J Gene Med 14(6):405-15. CAR-T cell therapy products have been approved for commercial use. Examples of commercially available CAR-T cell products that may be modified to incorporate an orthogonal receptor of the present invention include axicabtagene ciloleucel (marketed as Yescarta® commercially available from Gilead Pharmaceuticals) and tisagenlecleucel (marketed as Kymriah® commercially available from Novartis).

The antigen binding domain (ABD) of a CAR refers to a polypeptide that specifically binds to an antigen expressed on the surface of a target cell. The ABD may be any polypeptide that specifically binds to one or more antigens expressed on the surface of a target cell. In certain embodiments, the target cell antigen is a tumor antigen. Examples of tumor antigens that may be targeted by the ABD of a CAR include one or more antigens selected from the group including, but not limited to, the CD19, CD20, HER2, NY-ESO-1, MUC1, telomerase, CD123, FLT3, TIM3, CD99, CD96, B7-H3, CD33, IL1RAP, CLL1 (CLEC12A)PSA, CEA, VEGF, VEGF-R2, CD22, ROR1, mesothelin, c-Met, PSMA, Glycolipid F77, FAP, EGFRvIII, GD-2, MAGE A3 5T4, WT1, KG2D ligand (including MICA/B and ULBP-1, -2, -3, and -4), a folate receptor (FRa), and Wnt1 antigens.

“Comparable cell” shall mean a cell whose type is identical to that of another cell to which it is compared. Examples of comparable cells are cells from the same cell line.

“Inhibiting” the onset of a disorder shall mean either lessening the likelihood of the disorder's onset, or preventing the onset of the disorder entirely. In the preferred embodiment, inhibiting the onset of a disorder means preventing its onset entirely.

“Inhibiting” the expression of a gene in a cell shall mean either lessening the degree to which the gene is expressed, or preventing such expression entirely. “Specifically inhibit” the expression of a protein shall mean to inhibit that protein's expression (a) more than the expression of any other protein, or (b) more than the expression of all but 10 or fewer other proteins.

“Antibody” shall include, by way of example, both naturally occurring and non-naturally occurring antibodies. Specifically, this term includes polyclonal and monoclonal antibodies, and fragments thereof. Furthermore, this term includes chimeric antibodies and wholly synthetic antibodies, and fragments thereof.

“Anti-sense nucleic acid” shall mean any nucleic acid which, when introduced into a cell, specifically hybridizes to at least a portion of an mRNA in the cell encoding a protein (“target protein”) whose expression is to be inhibited, and thereby inhibits the target protein's expression.

“Specifically hybridize” to a nucleic acid shall mean, with respect to a first nucleic acid, that the first nucleic acid hybridizes to a second nucleic acid with greater affinity than to any other nucleic acid.

The terms “subject,” “individual,” and “patient” are used interchangeably herein to refer to a mammal being assessed for treatment and/or being treated. In an embodiment, the mammal is a human. The terms “subject,” “individual,” and “patient” thus encompass individuals having cancer. Subjects may be human, but also include other mammals, particularly those mammals useful as laboratory models for human disease, e.g. mouse, rat, etc.

“Suitable conditions” shall have a meaning dependent on the context in which this term is used. That is, when used in connection with an antibody, the term shall mean conditions that permit an antibody to bind to its corresponding antigen. When this term is used in connection with nucleic acid hybridization, the term shall mean conditions that permit a nucleic acid of at least 15 nucleotides in length to hybridize to a nucleic acid having a sequence complementary thereto. When used in connection with contacting an agent to a cell, this term shall mean conditions that permit an agent capable of doing so to enter a cell and perform its intended function. In one embodiment, the term “suitable conditions” as used herein means physiological conditions.

Treating may refer to any indicia of success in the treatment or amelioration or prevention of cancer including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with ***. The term “therapeutic effect” refers to the reduction, elimination, or prevention of the disease, symptoms of the disease, or side effects of the disease in the subject.

“In combination with”, “combination therapy” and “combination products” refer, in certain embodiments, to the concurrent administration to a patient of a first therapeutic (i.e., first therapeutic agent) and the compounds as used herein. When administered in combination, each component can be administered at the same time or sequentially in any order at different points in time. Thus, each component can be administered separately but sufficiently closely in time so as to provide the desired therapeutic effect.

“Concomitant administration” of a known therapeutic agent with a pharmaceutical composition of the present invention means administration of the therapeutic agent and inhibitor agent at such time that both the known therapeutic agent and the composition of the present invention will have a therapeutic effect. Such concomitant administration may involve concurrent (i.e. at the same time), prior, or subsequent administration of the drug with respect to the administration of a compound of the present invention. A person of ordinary skill in the art would have no difficulty determining the appropriate timing, sequence and dosages of administration for particular drugs and compositions of the present invention. Therapeutic agents contemplated for concomitant administration according to the methods of the present invention include any other agent for use in the treatment of cancer, particularly immune checkpoint inhibitors.

As used herein, the term “correlates,” or “correlates with,” and like terms, refers to a statistical association between instances of two events, where events include numbers, data sets, and the like. For example, when the events involve numbers, a positive correlation (also referred to herein as a “direct correlation”) means that as one increases, the other increases as well. A negative correlation (also referred to herein as an “inverse correlation”) means that as one increases, the other decreases.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

A “therapeutically effective amount” means the amount that, when administered to a subject for treating a disease, is sufficient to effect treatment for that disease.

The phrase “determining the treatment efficacy” and variants thereof can include any methods for determining that a treatment is providing a benefit to a subject. The term “treatment efficacy” and variants thereof are generally indicated by alleviation of one or more signs or symptoms associated with the disease and can be readily determined by one skilled in the art. “Treatment efficacy” may also refer to the prevention or amelioration of signs and symptoms of toxicities typically associated with standard or non-standard treatments of a disease. Determination of treatment efficacy is usually indication and disease specific and can include any methods known or available in the art for determining that a treatment is providing a beneficial effect to a patient. For example, evidence of treatment efficacy can include but is not limited to remission of the disease or indication. Further, treatment efficacy can also include general improvements in the overall health of the subject, such as but not limited to enhancement of patient life quality, increase in predicted subject survival rate, decrease in depression or decrease in rate of recurrence of the indication (increase in remission time). (See, e.g., Physicians' Desk Reference (2010).)

An “immune cold” tumor, also known as an “immunologically cold” tumor, refers to a type of cancer that is characterized by a lack of immune cell infiltration, particularly T cells, into the tumor microenvironment. These tumors exhibit low levels of immune activity and are often resistant to immunotherapies, such as immune checkpoint inhibitors, which rely on the presence of an active immune response to be effective. Key features of immune cold tumors include: low T cell infiltration, with few or no T cells within the tumor microenvironment, indicating a lack of immune recognition and attack on the cancer cells. Low expression of immune-related genes, where these tumors often show low expression of genes associated with immune activation, such as those coding for cytokines, chemokines, and other molecules involved in immune cell recruitment and activation. Poor immunogenicity, where immune cold tumors may have a lower mutational burden, meaning they produce fewer neoantigens that can be recognized by the immune system. Immune evasion mechanisms, where these tumors might employ various strategies to evade immune detection, such as producing immunosuppressive molecules, altering antigen presentation, or creating a physical barrier that prevents immune cells from entering the tumor. Because of these characteristics, immune cold tumors are challenging to treat with current immunotherapies, which have been more successful in “immune hot” tumors-tumors with significant immune cell infiltration and high levels of immune activation.

### Combination Therapies

A KDM2A inhibitor may be administered in a combination therapy with an effective dose or doses of additional immune regulatory agent(s), e.g. immune checkpoint inhibitors that reverse the inhibition of immune responses through administering antagonists of inhibitory signals, agonists of immune costimulatory molecules to increase responsiveness; CAR-T cell therapy and other adoptive cellular therapies, such as TIL (expanded tumor-infiltrating lymphocytes), NK cells, macrophages, B cells, etc. In some embodiments a synergistic response is observed, relative to the level of anti-tumor activity observed with either agent administered singly. The dose of immune checkpoint inhibitor, for example, may be equal to, or less than the effective dose administered in the absence of the inhibitor.

Immune-checkpoint receptors that have been most actively studied in the context of clinical cancer immunotherapy, cytotoxic T-lymphocyte-associated antigen 4 (CTLA4; also known as CD152) and programmed cell death protein 1 (PD1; also known as CD279)—are both inhibitory receptors. The clinical activity of antibodies that block either of these receptors implies that antitumor immunity can be enhanced at multiple levels and that combinatorial strategies can be intelligently designed, guided by mechanistic considerations and preclinical models.

CTLA4 is expressed exclusively on T cells where it primarily regulates the amplitude of the early stages of T cell activation. CTLA4 counteracts the activity of the T cell co-stimulatory receptor, CD28. CD28 and CTLA4 share identical ligands: CD80 (also known as B7.1) and CD86 (also known as B7.2). The major physiological roles of CTLA4 are downmodulation of helper T cell activity and enhancement of regulatory T (TReg) cell immunosuppressive activity. CTLA4 blockade results in a broad enhancement of immune responses. Two fully humanized CTLA4 antibodies, ipilimumab and tremelimumab, are in clinical testing and use. Clinically the response to immune-checkpoint blockers is slow and, in many patients, delayed up to 6 months after treatment initiation. In some cases, metastatic lesions actually increase in size on computed tomography (CT) or magnetic resonance imaging (MRI) scans before regressing. Anti-CTLA4 antibodies that antagonize this inhibitory immune function are very potent therapeutics but have significant side effects since this enables T cell activity against the self that is usually inhibited through these inhibitory molecules and pathways.

In some embodiments the dose of anti-CTLA4 agent administered in a combination therapy is reduced to a level that minimizes undesirable side effects, e.g. at a dose that is up to about 90% of the currently approved dose, that is up to about 80%, up to about 70%, up to about 60%, up to about 50%, up to about 40%, up to about 30%, up to about 20%, up to about 10%, up to about 5% of a conventional dose. In some embodiments the number of doses is reduced, e.g. dosing with anti-CTLA4 agent not more than 1×, not more than 2×, not more than 3×, etc. As a reference, for example, current protocols usually call for administration of ipilimumab at a dose of 3 mg/kg, administered every 3 weeks for a total of 4 doses; optionally in combination with additional agents such as, for example dacarbazine or temozolomide; or with peptide vaccines. Other protocols have explored administration of ipilimumab at the dose of 10 mg/kg as a single agent against metastatic melanoma.

Tremelimumab has been administered as a single antibody infusion at doses ranging from 0.01 mg/kg to 15 mg/kg. Objective responses were evident at doses of 3 mg/kg and above. The majority of responses were noted in patients that achieved sustained plasma levels of tremelimumab beyond 30 μg/ml at one month. The doses of 10 mg/kg administered every month and 15 mg/kg administered every 3 months have been studied further in a phase II randomized clinical trial, however toxicity was doubled when dosing more frequently with the 10 mg/kg monthly regimen. Based on these data, single agent tremelimumab at 15 mg/kg every 3 months was chosen for clinical trials.

For some patients, the ability of CTLA-4 blockade to activate the immune system results in inflammatory manifestations characterized as immune-related adverse events (irAEs). The most clinically significant irAE is enterocolitis which can range in severity; grade Ill/IV enterocolitis is seen in ~15% of patients treated with ipilimumab at 10 mg/kg. Additional irAEs include rash/pruritus (>50%), hepatitis (5-10%), hypophysitis (5%), uveitis (<2%), pancreatitis (<2%), and leucopenia (<2%). The combination of agents may reduce such adverse events. In some embodiments of the invention a method is provided for treating cancer with a combination of an agents, where the dosing of agents in the combination provides for a treatment of cancer with a clinically significant reduction in immune-related adverse events relative to the dosing required for anti-CTLA-4 in the absence of the inhibitor.

Other immune-checkpoint proteins are PD1 and PDL1. Three anti-PD-1 antibodies have been approved by the FDA: pembrolizumab (Keytruda), nivolumab (Opdivo), and cemiplimab (Libtayo). Anti-PD1 agents in clinical trials include, for example, JTX-4014; Spartalizumab (PDR001); Camrelizumab (SHR1210); Sintilimab (IBI308); Tislelizumab (BGB-A317) is a humanized IgG4 anti-PD-1 monoclonal antibody; Toripalimab (JS 001) is a humanized IgG4 monoclonal antibody against PD-1; INCMGA00012 (MGA012) is a humanized IgG4 monoclonal antibody; AMP-224; AMP-514 (MED10680).

The major role of PD1 is to limit the activity of T cells in peripheral tissues at the time of an inflammatory response to infection and to limit autoimmunity. PD1 expression is induced when T cells become activated. When engaged by one of its ligands, PD1 inhibits kinases that are involved in T cell activation. PD1 is highly expressed on T_{Reg} cells, where it may enhance their proliferation in the presence of ligand. Because many tumors are highly infiltrated with T_{Reg} cells, blockade of the PD1 pathway may also enhance antitumor immune responses by diminishing the number and/or suppressive activity of intratumoral T_{Reg} cells.

The two ligands for PD1 are PD1 ligand 1 (PDL1; also known as B7-H1 and CD274) and PDL2 (also known as B7-DC and CD273). Approved for clinical use are Atezolizumab (Tecentriq) is a fully humanised IgG1 (immunoglobulin 1) antibody; Avelumab (Bavencio) is a fully human IgG1 antibody; Durvalumab (Imfinzi) is a fully human IgG1 antibody. PD-L1 inhibitors in clinical trials include KN035 with subcutaneous formulation; CK-301; AUNP12; CA-170; BMS-986189.

PD1 ligands are commonly upregulated on the tumor cell surface from many different human tumors. On cells from solid tumors, the major PD1 ligand that is expressed is PDL1. PDL1 is expressed on cancer cells and through binding to its receptor PD1 on T cells it inhibits T cell activation/function. Therefore, PD1 and PDL1 blocking agents can overcome this inhibitory signaling and maintain or restore anti-tumor T cell function. However, since PDL1 is expressed on tumor cells, antibodies that bind and block PDL1 can also enable ADCP, ADCC, and CDC of tumor cells. Thus a combination of anti-PDL1 agents with the inhibitor can enhance the anti-tumor potency. These agents may be administered together (over the same course of treatment, not necessarily the same day and frequency).

Lymphocyte activation gene 3 (LAG3; also known as CD223), 2B4 (also known as CD244), B and T lymphocyte attenuator (BTLA; also known as CD272), T cell membrane protein 3 (TIM3; also known as HAVcr2), adenosine A2a receptor (A2aR) and the family of killer inhibitory receptors have each been associated with the inhibition of lymphocyte activity and in some cases the induction of lymphocyte anergy. Antibody targeting of these receptors can be used in the methods of the invention.

LAG3 is a CD4 homolog that enhances the function of T_{Reg} cells. LAG3 also inhibits CD8⁺ effector T cell functions independently of its role on T_{Reg} cells. The only known ligand for LAG3 is MHC class II molecules, which are expressed on tumor-infiltrating macrophages and dendritic cells. LAG3 is one of various immune-checkpoint receptors that are coordinately upregulated on both T_{Reg} cells and anergic T cells, and simultaneous blockade of these receptors can result in enhanced reversal of this anergic state relative to blockade of one receptor alone. In particular, PD1 and LAG3 are commonly co-expressed on anergic or exhausted T cells. Dual blockade of LAG3 and PD1 synergistically reversed anergy among tumor-specific CD8⁺ T cells and virus-specific CD8⁺ T cells in the setting of chronic infection. LAG3 blocking agents can overcome this inhibitory signaling and maintain or restore anti-tumor T cell function.

TIM3 inhibits T helper 1 (T_{H}1) cell responses, and TIM3 antibodies enhance antitumor immunity. TIM3 has also been reported to be co-expressed with PD1 on tumor-specific CD8⁺ T cells. Tim3 blocking agents can overcome this inhibitory signaling and maintain or restore anti-tumor T cell function.

BTLA is an inhibitory receptor on T cells that interacts with TNFRSF14. BTLA^{hi} T cells are inhibited in the presence of its ligand. The system of interacting molecules is complex: CD160 (an immunoglobulin superfamily member) and LIGHT (also known as TNFSF14), mediate inhibitory and co-stimulatory activity, respectively. Signaling can be bidirectional, depending on the specific combination of interactions. Dual blockade of BTLA and PD1 enhances antitumor immunity.

A2aR, the ligand of which is adenosine, inhibits T cell responses, in part by driving CD4⁺ T cells to express FOXP3 and hence to develop into T_{Reg} cells. Deletion of this receptor results in enhanced and sometimes pathological inflammatory responses to infection. A2aR can be inhibited either by antibodies that block adenosine binding or by adenosine analogues.

Agents that agonize an immune costimulatory molecule are also useful in the methods of the invention. Such agents include agonists or CD40 and OX40. CD40 is a costimulatory protein found on antigen presenting cells (APCs) and is required for their activation. These APCs include phagocytes (macrophages and dendritic cells) and B cells. CD40 is part of the TNF receptor family. The primary activating signaling molecules for CD40 are IFNγ and CD40 ligand (CD40L). Stimulation through CD40 activates macrophages. Agonistic CD40 agents may be administered substantially simultaneously with the inhibitor; or may be administered prior to and concurrently with treatment.

OX40 (CD134) is a member of the TNFR super-family and expressed on T cells. Molecules that bind OX40 can stimulate proliferation and differentiation of T cells.

Other immuno-oncology agents that can be administered in combination according to the methods described herein include antibodies specific for chemokine receptors, including without limitation anti-CCR4 and anti-CCR2. Anti CCR4 (CD194) antibodies of interest include humanized monoclonal antibodies directed against C-C chemokine receptor 4 (CCR4) with potential anti-inflammatory and antineoplastic activities. Exemplary is mogamulizumab, which selectively binds to and blocks the activity of CCR4, which may inhibit CCR4-mediated signal transduction pathways and, so, chemokine-mediated cellular migration and proliferation of T cells, and chemokine-mediated angiogenesis. In addition, this agent may induce antibody-dependent cell-mediated cytotoxicity (ADCC) against CCR4-positive T cells. CCR4, a G-coupled-protein receptor for C-C chemokines such MIP-1, RANTES, TARC and MCP-1, is expressed on the surfaces of some types of T cells, endothelial cells, and some types of neurons. CCR4, also known as CD194, may be overexpressed on adult T-cell lymphoma (ATL) and peripheral T-cell lymphoma (PTCL) cells.

Anti-CCR4 Ab may be administered in combination with an agent for CD47 blockade for enhanced depletion of CCR4 positive target cells, including without limitation T-cell lymphoma, especially cutaneous T cell lymphoma (CTCL), or DLBCL, breast cancer, renal cell carcinoma, colon cancer, other. CD47 blockade can synergize with cancer targeting monoclonal antibodies and enhance their efficacy for ADCP and ADCC.

Anti-CCR2 (CD192) Ab. CCR2 is expressed on inflammatory macrophages that can be found in various inflammatory conditions, e.g. rheumatoid arthritis; and have also been identified as expressed on tumor promoting macrophages. Chemokines that bind to CCR2, e.g. CCL2, can recruit and activate the inflammatory macrophages. Inhibiting the chemokine signaling through CCR2 with anti-CCR2 antibodies may result in lower frequencies of undesirable autoimmune or tumor promoting macrophages through inhibition of recruiting or antibody dependent depletion, resulting in mitigation of autoimmune diseases like rheumatoid arthritis, or inhibition of tumor growth or metastasis. CCR2 is also expressed on regulatory T cells, and the CCR2 ligand, CCL2, mediates recruitment of regulatory T cells into tumors. Regulatory T cells suppress a response for anti-tumor T cells and thus their inhibition or depletion is desired. Anti-CCR2 Ab is administered in combination for enhanced depletion of CCR2 positive inflammatory and tumor promoting macrophages and regulatory T cells. Inflammatory (tumor associated macrophages) and regulatory T cells suppress an anti-tumor immune response and therefore their inhibition or depletion is desired.

Chemotherapy combinations may include treatment with Abitrexate (Methotrexate Injection), Abraxane (Paclitaxel Injection), Adcetris (Brentuximab Vedotin Injection), Adriamycin (Doxorubicin), Adrucil Injection (5-FU (fluorouracil)), Afinitor (Everolimus), Afinitor Disperz (Everolimus), Alimta (PEMET EXED), Alkeran Injection (Melphalan Injection), Alkeran Tablets (Melphalan), Aredia (Pamidronate), Arimidex (Anastrozole), Aromasin (Exemestane), Arranon (Nelarabine), Arzerra (Ofatumumab Injection), Avastin (Bevacizumab), Bexxar (Tositumomab), BiCNU (Carmustine), Blenoxane (Bleomycin), Bosulif (Bosutinib), Busulfex Injection (Busulfan Injection), Campath (Alemtuzumab), Camptosar (Irinotecan), Caprelsa (Vandetanib), Casodex (Bicalutamide), CeeNU (Lomustine), CeeNU Dose Pack (Lomustine), Cerubidine (Daunorubicin), Clolar (Clofarabine Injection), Cometriq (Cabozantinib), Cosmegen (Dactinomycin), CytosarU (Cytarabine), Cytoxan (Cytoxan), Cytoxan Injection (Cyclophosphamide Injection), Dacogen (Decitabine), DaunoXome (Daunorubicin Lipid Complex Injection), Decadron (Dexamethasone), DepoCyt (Cytarabine Lipid Complex Injection), Dexamethasone Intensol (Dexamethasone), Dexpak Taperpak (Dexamethasone), Docefrez (Docetaxel), Doxil (Doxorubicin Lipid Complex Injection), Droxia (Hydroxyurea), DTIC (Decarbazine), Eligard (Leuprolide), Ellence (Ellence (epirubicin)), Eloxatin (Eloxatin (oxaliplatin)), Elspar (Asparaginase), Emcyt (Estramustine), Erbitux (Cetuximab), Erivedge (Vismodegib), Erwinaze (Asparaginase Erwinia chrysanthemi), Ethyol (Amifostine), Etopophos (Etoposide Injection), Eulexin (Flutamide), Fareston (Toremifene), Faslodex (Fulvestrant), Femara (Letrozole), Firmagon (Degarelix Injection), Fludara (Fludarabine), Folex (Methotrexate Injection), Folotyn (Pralatrexate Injection), FUDR (FUDR (floxuridine)), Gemzar (Gemcitabine), Gilotrif (Afatinib), Gleevec (Imatinib Mesylate), Gliadel Wafer (Carmustine wafer), Halaven (Eribulin Injection), Herceptin (Trastuzumab), Hexalen (Altretamine), Hycamtin (Topotecan), Hycamtin (Topotecan), Hydrea (Hydroxyurea), Iclusig (Ponatinib), Idamycin PFS (Idarubicin), Ifex (Ifosfamide), Inlyta (Axitinib), Intron A alfab (Interferon alfa-2a), Iressa (Gefitinib), Istodax (Romidepsin Injection), Ixempra (Ixabepilone Injection), Jakafi (Ruxolitinib), Jevtana (Cabazitaxel Injection), Kadcyla (Ado-trastuzumab Emtansine), Kyprolis (Carfilzomib), Leukeran (Chlorambucil), Leukine (Sargramostim), Leustatin (Cladribine), Lupron (Leuprolide), Lupron Depot (Leuprolide), Lupron DepotPED (Leuprolide), Lysodren (Mitotane), Marqibo Kit (Vincristine Lipid Complex Injection), Matulane (Procarbazine), Megace (Megestrol), Mekinist (Trametinib), Mesnex (Mesna), Mesnex (Mesna Injection), Metastron (Strontium-89 Chloride), Mexate (Methotrexate Injection), Mustargen (Mechlorethamine), Mutamycin (Mitomycin), Myleran (Busulfan), Mylotarg (Gemtuzumab Ozogamicin), Navelbine (Vinorelbine), Neosar Injection (Cyclophosphamide Injection), Neulasta (filgrastim), Neulasta (pegfilgrastim), Neupogen (filgrastim), Nexavar (Sorafenib), Nilandron (Nilandron (nilutamide)), Nipent (Pentostatin), Nolvadex (Tamoxifen), Novantrone (Mitoxantrone), Oncaspar (Pegaspargase), Oncovin (Vincristine), Ontak (Denileukin Diftitox), Onxol (Paclitaxel Injection), Panretin (Alitretinoin), Paraplatin (Carboplatin), Perjeta (Pertuzumab Injection), Platinol (Cisplatin), Platinol (Cisplatin Injection), PlatinolAQ (Cisplatin), PlatinolAQ (Cisplatin Injection), Pomalyst (Pomalidomide), Prednisone Intensol (Prednisone), Proleukin (Aldesleukin), Purinethol (Mercaptopurine), Reclast (Zoledronic acid), Revlimid (Lenalidomide), Rheumatrex (Methotrexate), Rituxan (Rituximab), RoferonA alfaa (Interferon alfa-2a), Rubex (Doxorubicin), Sandostatin (Octreotide), Sandostatin LAR Depot (Octreotide), Soltamox (Tamoxifen), Sprycel (Dasatinib), Sterapred (Prednisone), Sterapred DS (Prednisone), Stivarga (Regorafenib), Supprelin LA (Histrelin Implant), Sutent (Sunitinib), Sylatron (Peginterferon Alfa-2b Injection (Sylatron)), Synribo (Omacetaxine Injection), Tabloid (Thioguanine), Taflinar (Dabrafenib), Tarceva (Erlotinib), Targretin Capsules (Bexarotene), Tasigna (Decarbazine), Taxol (Paclitaxel Injection), Taxotere (Docetaxel), Temodar (Temozolomide), Temodar (Temozolomide Injection), Tepadina (Thiotepa), Thalomid (Thalidomide), TheraCys BCG (BCG), Thioplex (Thiotepa), TICE BCG (BCG), Toposar (Etoposide Injection), Torisel (Temsirolimus), Treanda (Bendamustine hydrochloride), Trelstar (Triptorelin Injection), Trexall (Methotrexate), Trisenox (Arsenic trioxide), Tykerb (lapatinib), Valstar (Valrubicin Intravesical), Vantas (Histrelin Implant), Vectibix (Panitumumab), Velban (Vinblastine), Velcade (Bortezomib), Vepesid (Etoposide), Vepesid (Etoposide Injection), Vesanoid (Tretinoin), Vidaza (Azacitidine), Vincasar PFS (Vincristine), Vincrex (Vincristine), Votrient (Pazopanib), Vumon (Teniposide), Wellcovorin IV (Leucovorin Injection), Xalkori (Crizotinib), Xeloda (Capecitabine), Xtandi (Enzalutamide), Yervoy (Ipilimumab Injection), Zaltrap (Ziv-aflibercept Injection), Zanosar (Streptozocin), Zelboraf (Vemurafenib), Zevalin (lbritumomab Tiuxetan), Zoladex (Goserelin), Zolinza (Vorinostat), Zometa (Zoledronic acid), Zortress (Everolimus), Zytiga (Abiraterone), Nimotuzumab and immune checkpoint inhibitors such as nivolumab, pembrolizumab/MK-3475, pidilizumab and AMP-224 targeting PD-1; and BMS-935559, MED14736, MPDL3280A and MSB0010718C targeting PD-L1 and those targeting CTLA-4 such as ipilimumab.

Radiotherapy means the use of radiation, usually X-rays, to treat illness. X-rays were discovered in 1895 and since then radiation has been used in medicine for diagnosis and investigation (X-rays) and treatment (radiotherapy). Radiotherapy may be from outside the body as external radiotherapy, using X-rays, cobalt irradiation, electrons, and more rarely other particles such as protons. It may also be from within the body as internal radiotherapy, which uses radioactive metals or liquids (isotopes) to treat cancer.

A number of antibodies that target tumor cell antigens are currently in clinical use for the treatment of cancer, and others are in varying stages of clinical development. For example, there are a number of antigens and corresponding monoclonal antibodies for the treatment of B cell malignancies. One target antigen is CD20. Rituximab is a chimeric unconjugated monoclonal antibody directed at the CD20 antigen. CD20 has an important functional role in B cell activation, proliferation, and differentiation. The CD52 antigen is targeted by the monoclonal antibody alemtuzumab, which is indicated for treatment of chronic lymphocytic leukemia. CD22 is targeted by a number of antibodies, and has recently demonstrated efficacy combined with toxin in chemotherapy-resistant hairy cell leukemia. Two new monoclonal antibodies targeting CD20, tositumomab and ibritumomab, have been submitted to the Food and Drug Administration (FDA). These antibodies are conjugated with radioisotopes. Alemtuzumab (Campath) is used in the treatment of chronic lymphocytic leukemia; Gemtuzumab (Mylotarg) finds use in the treatment of acute myelogenous leukemia; Ibritumomab (Zevalin) finds use in the treatment of non-Hodgkin's lymphoma; Panitumumab (Vectibix) finds use in the treatment of colon cancer.

The CD52 antigen is targeted by the monoclonal antibody alemtuzumab, which is indicated for treatment of chronic lymphocytic leukemia; colon cancer and lung cancer. CD22 is targeted by a number of antibodies, and has recently demonstrated efficacy combined with toxin in chemotherapy-resistant hairy cell leukemia.

Gemtuzumab (Mylotarg) finds use in the treatment of acute myelogenous leukemia; Ibritumomab (Zevalin) finds use in the treatment of non-Hodgkin's lymphoma; Panitumumab (Vectibix) finds use in the treatment of colon cancer.

Cetuximab (Erbitux) is also of interest for use in the methods of the invention. The antibody binds to the EGF receptor (EGFR), and has been used in the treatment of solid tumors including colon cancer and squamous cell carcinoma of the head and neck.

Monoclonal antibodies useful in the methods of the invention that have been used in solid tumors include, without limitation, edrecolomab and trastuzumab (herceptin). Edrecolomab targets the 17-1A antigen seen in colon and rectal cancer, and has been approved for use in Europe for these indications. Trastuzumab targets the HER-2/neu antigen. This antigen is seen on 25% to 35% of breast cancers. Cetuximab (Erbitux) is also of interest for use in the methods of the invention. The antibody binds to the EGF receptor (EGFR), and has been used in the treatment of solid tumors including colon cancer and squamous cell carcinoma of the head and neck.

The terms “cancer,” “neoplasm,” and “tumor” are used interchangeably herein to refer to cells which exhibit autonomous, unregulated growth, such that they exhibit an aberrant growth phenotype characterized by a significant loss of control over cell proliferation. Cells of interest for detection, analysis, or treatment in the present application include precancerous (e.g., benign), malignant, pre-metastatic, metastatic, and non-metastatic cells. Cancers of virtually every tissue are known. The phrase “cancer burden” refers to the quantum of cancer cells or cancer volume in a subject. Reducing cancer burden accordingly refers to reducing the number of cancer cells or the cancer volume in a subject. The term “cancer cell” as used herein refers to any cell that is a cancer cell or is derived from a cancer cell e.g. clone of a cancer cell.

Many types of cancers are known to those of skill in the art, including solid tumors such as carcinomas, sarcomas, glioblastomas, melanomas, lymphomas, myelomas, etc., and circulating cancers such as leukemias. Examples of cancer include but are not limited to, ovarian cancer, breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, and brain cancer.

The types of cancer that can be treated using the subject methods of the present invention include but are not limited to adrenal cortical cancer, anal cancer, aplastic anemia, bile duct cancer, bladder cancer, bone cancer, bone metastasis, brain cancers, central nervous system (CNS) cancers, peripheral nervous system (PNS) cancers, breast cancer, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's lymphoma, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcomas, melanoma skin cancer, non-melanoma skin cancers, stomach cancer, testicular cancer, thymus cancer, thyroid cancer, uterine cancer (e.g. uterine sarcoma), transitional cell carcinoma, vaginal cancer, vulvar cancer, mesothelioma, squamous cell or epidermoid carcinoma, bronchial adenoma, choriocarinoma, head and neck cancers, teratocarcinoma, or Waldenstrom's macroglobulinemia.

In specific embodiments, a cancer selected for treatment is head and neck squamous cell carcinoma (HNSCC).

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

As used herein, the terms “cancer recurrence” and “tumor recurrence,” and grammatical variants thereof, refer to further growth of neoplastic or cancerous cells after diagnosis of cancer. Particularly, recurrence may occur when further cancerous cell growth occurs in the cancerous tissue. “Tumor spread,” similarly, occurs when the cells of a tumor disseminate into local or distant tissues and organs; therefore tumor spread encompasses tumor metastasis. “Tumor invasion” occurs when the tumor growth spread out locally to compromise the function of involved tissues by compression, destruction, or prevention of normal organ function.

As used herein, the term “metastasis” refers to the growth of a cancerous tumor in an organ or body part, which is not directly connected to the organ of the original cancerous tumor. Metastasis will be understood to include micrometastasis, which is the presence of an undetectable amount of cancerous cells in an organ or body part which is not directly connected to the organ of the original cancerous tumor. Metastasis can also be defined as several steps of a process, such as the departure of cancer cells from an original tumor site, and migration and/or invasion of cancer cells to other parts of the body.

The term “sample” with respect to a patient encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents; washed; or enrichment for certain cell populations, such as cancer cells. The definition also includes sample that have been enriched for particular types of molecules, e.g., nucleic acids, polypeptides, etc. The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient's cancer cell, e.g., a sample comprising polynucleotides and/or polypeptides that is obtained from a patient's cancer cell (e.g., a cell lysate or other cell extract comprising polynucleotides and/or polypeptides); and a sample comprising cancer cells from a patient. A biological sample comprising a cancer cell from a patient can also include non-cancerous cells.

The term “diagnosis” is used herein to refer to the identification of a molecular or pathological state, disease or condition, such as the identification of a molecular subtype of breast cancer, prostate cancer, or other type of cancer.

The term “prognosis” is used herein to refer to the prediction of the likelihood of cancer-attributable death or progression, including recurrence, metastatic spread, and drug resistance, of a neoplastic disease, such as ovarian cancer. The term “prediction” is used herein to refer to the act of foretelling or estimating, based on observation, experience, or scientific reasoning. In one example, a physician may predict the likelihood that a patient will survive, following surgical removal of a primary tumor and/or chemotherapy for a certain period of time without cancer recurrence.

As used herein, endpoints for treatment will be given a meaning as known in the art and as used by the Food and Drug Administration.

Overall survival is defined as the time from randomization until death from any cause, and is measured in the intent-to-treat population. Survival is considered the most reliable cancer endpoint, and when studies can be conducted to adequately assess survival, it is usually the preferred endpoint. This endpoint is precise and easy to measure, documented by the date of death. Bias is not a factor in endpoint measurement. Survival improvement should be analyzed as a risk-benefit analysis to assess clinical benefit. Overall survival can be evaluated in randomized controlled studies. Demonstration of a statistically significant improvement in overall survival can be considered to be clinically significant if the toxicity profile is acceptable, and has often supported new drug approval. A benefit of the methods of the invention can include increased overall survival of patients.

Endpoints that are based on tumor assessments include DFS, ORR, TTP, PFS, and time-to-treatment failure (TTF). The collection and analysis of data on these time-dependent endpoints are based on indirect assessments, calculations, and estimates (e.g., tumor measurements). Disease-Free Survival (DFS) is defined as the time from randomization until recurrence of tumor or death from any cause. The most frequent use of this endpoint is in the adjuvant setting after definitive surgery or radiotherapy. DFS also can be an important endpoint when a large percentage of patients achieve complete responses with chemotherapy.

Objective Response Rate. ORR is defined as the proportion of patients with tumor size reduction of a predefined amount and for a minimum time period. Response duration usually is measured from the time of initial response until documented tumor progression. Generally, the FDA has defined ORR as the sum of partial responses plus complete responses. When defined in this manner, ORR is a direct measure of drug antitumor activity, which can be evaluated in a single-arm study.

Time to Progression and Progression-Free Survival. TTP and PFS have served as primary endpoints for drug approval. TTP is defined as the time from randomization until objective tumor progression; TTP does not include deaths. PFS is defined as the time from randomization until objective tumor progression or death. The precise definition of tumor progression is important and should be carefully detailed in the protocol.

As used herein, the term “correlates,” or “correlates with,” and like terms, refers to a statistical association between instances of two events, where events include numbers, data sets, and the like. For example, when the events involve numbers, a positive correlation (also referred to herein as a “direct correlation”) means that as one increases, the other increases as well. A negative correlation (also referred to herein as an “inverse correlation”) means that as one increases, the other decreases.

### Methods of Use

Methods are provided for treating or reducing primary or metastatic cancer in a regimen comprising contacting the targeted cancer cells with a combination of (i) an effective dose of an inhibitor of KDM2A; and (ii) an effective dose of one or more of an agent that agonizes an immune costimulatory molecule, e.g. CD40, OX40, etc.; and/or (iii) an effective dose of an agent that antagonizes an immune inhibitory molecule, e.g. CTLA-4, PD1, PDL1, etc. Such methods include administering to a subject in need of treatment a therapeutically effective amount or an effective dose of the combined agents of the invention, including without limitation combinations of the reagent with a chemotherapeutic drug, radiation therapy, etc. Alternatively the combined agents comprise administration of an adoptive cell therapy, including administration of CART or CAR-NK cells, as disclosed herein. In some embodiments the targeted cancer is a head an neck squamous cell carcinoma. In some embodiments the cancer is determined deficient in expression or activity of NSD1. In some embodiments the cancer is creened prior to treatment for a deficiency in expression or activity of NSD1.

Effective doses of the combined agents of the present invention for the treatment of cancer vary depending upon many different factors, including means of administration, target site, physiological state of the patient, whether the patient is human or an animal, other medications administered, and whether treatment is prophylactic or therapeutic. Usually, the patient is a human, but nonhuman mammals may also be treated, e.g. companion animals such as dogs, cats, horses, etc., laboratory mammals such as rabbits, mice, rats, etc., and the like. Treatment dosages can be titrated to optimize safety and efficacy.

In some embodiments, the therapeutic dosage of each agent may range from about 0.0001 to 100 mg/kg, and more usually 0.01 to 5 mg/kg, of the host body weight. For example dosages can be 1 mg/kg body weight or 10 mg/kg body weight or within the range of 1-10 mg/kg. An exemplary treatment regime entails administration once every two weeks or once a month or once every 3 to 6 months. Therapeutic entities of the present invention are usually administered on multiple occasions. Intervals between single dosages can be weekly, monthly or yearly. Intervals can also be irregular as indicated by measuring blood levels of the therapeutic entity in the patient. Alternatively, therapeutic entities of the present invention can be administered as a sustained release formulation, in which case less frequent administration is required. Dosage and frequency vary depending on the half-life of the polypeptide in the patient.

In still other embodiments, methods of the present invention include treating, reducing or preventing tumor growth, tumor metastasis or tumor invasion of cancers including carcinomas, hematologic cancers, melanomas, sarcomas, gliomas, etc.

Compositions for the treatment of cancer can be administered by parenteral, topical, intravenous, intratumoral, oral, subcutaneous, intraarterial, intracranial, intraperitoneal, intranasal or intramuscular means. A typical route of administration is intravenous or intratumoral, although other routes can be equally effective.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

Toxicity of the combined agents described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition.

The pharmaceutical compositions can be administered in a variety of unit dosage forms depending upon the method of administration. For example, unit dosage forms suitable for oral administration include, but are not limited to, powder, tablets, pills, capsules and lozenges. It is recognized that compositions of the invention when administered orally, should be protected from digestion. This is typically accomplished either by complexing the molecules with a composition to render them resistant to acidic and enzymatic hydrolysis, or by packaging the molecules in an appropriately resistant carrier, such as a liposome or a protection barrier. Means of protecting agents from digestion are well known in the art.

The compositions for administration will commonly be dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, e.g., sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patient's needs (e.g., Remington's Pharmaceutical Science (15th ed., 1980) and Goodman & Gillman, The Pharmacological Basis of Therapeutics (Hardman et al., eds., 1996)).

Also within the scope of the invention are kits comprising the active agents and formulations thereof, of the invention and instructions for use. The kit can further contain a least one additional reagent, e.g. a chemotherapeutic drug, etc. Kits typically include a label indicating the intended use of the contents of the kit. The term label includes any writing, or recorded material supplied on or with the kit, or which otherwise accompanies the kit.

The compositions can be administered for therapeutic treatment. Compositions are administered to a patient in an amount sufficient to substantially reduce the number and/or viability of targeted cells, as described above. An amount adequate to accomplish this is defined as a “therapeutically effective dose.”, which may provide for an improvement in overall survival rates. Single or multiple administrations of the compositions may be administered depending on the dosage and frequency as required and tolerated by the patient. The particular dose required for a treatment will depend upon the medical condition and history of the mammal, as well as other factors such as age, weight, gender, administration route, efficiency, etc.

The agents may be administered one or a plurality of days, and in some embodiments is administered daily, every two days, semi-weekly, weekly, etc. for a period of from about 1, about 2, about 3, about 4, about 5, about 6, about 7 or more weeks, up to a chronic maintenance level of dosing. Therapeutic entities of the present invention are usually administered on multiple occasions. Intervals between single dosages can be weekly, monthly or yearly. Intervals can also be irregular as indicated by measuring blood levels of the therapeutic entity in the patient. Alternatively, therapeutic entities of the present invention can be administered as a sustained release formulation, in which case less frequent administration is required. Dosage and frequency vary depending on the half-life of the polypeptide in the patient.

For therapeutic applications, therapeutic entities of the present invention are administered to a patient suspected of, or already suffering from such a disease in an amount sufficient to cure, or at least partially arrest, the symptoms of the disease (biochemical, histologic and/or behavioral), including its complications and intermediate pathological phenotypes in development of the disease. An amount adequate to accomplish therapeutic or prophylactic treatment is defined as a therapeutically- or prophylactically-effective dose. In both prophylactic and therapeutic regimes, agents are usually administered in several dosages until a sufficient response has been achieved.

According to the present invention, compositions can be administered by parenteral, topical, intravenous, oral, subcutaneous, intraarterial, intracranial, intraperitoneal, intranasal, aerosol, or intramuscular means. The most typical route of administration is intravenous although other routes can be equally effective.

For parenteral administration, compositions of the invention can be administered as injectable dosages of a solution or suspension of the substance in a physiologically acceptable diluent with a pharmaceutical carrier that can be a sterile liquid such as water, oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. Antibodies and/or polypeptides can be administered in the form of a depot injection or implant preparation which can be formulated in such a manner as to permit a sustained release of the active ingredient. An exemplary composition comprises polypeptide at 1 mg/mL, formulated in aqueous buffer consisting of 10 mM Tris, 210 mM sucrose, 51 mM L-arginine, 0.01% polysorbate 20, adjusted to pH 7.4 with HCl or NaOH.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, and transdermal applications.

For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and contain 10%-95% of active ingredient, preferably 25%-70%.

The pharmaceutical compositions are generally formulated as sterile, substantially isotonic and in full compliance with all Good Manufacturing Practice (GMP) regulations of the U.S. Food and Drug Administration. Preferably, a therapeutically effective dose will provide therapeutic benefit without causing substantial toxicity.

Toxicity of the proteins described herein can be determined by standard pharmaceutical procedures in cell cultures or experimental animals, e.g., by determining the LD50 (the dose lethal to 50% of the population) or the LD100 (the dose lethal to 100% of the population). The dose ratio between toxic and therapeutic effect is the therapeutic index. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition. (See, e.g., Fingl et al., 1975, In: The Pharmacological Basis of Therapeutics, Ch. 1).

Also within the scope of the invention are kits comprising the compositions of the invention and instructions for use. The kit can further contain a least one additional reagent. Kits typically include a label indicating the intended use of the contents of the kit. The term label includes any writing, or recorded material supplied on or with the kit, or which otherwise accompanies the kit.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. It is also understood that the terminology used herein is for the purposes of describing particular embodiments

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or only and is not intended to limit the scope of the present invention which will be limited only by the appended claims.

Those skilled in the art will recognize, or be able to ascertain using no more than routine experimentation, many equivalents to the specific embodiments of the invention described herein. Such equivalents are intended to be encompassed by the appended claims.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Targeting KDM2A Enhances T Cell Infiltration in NSD1-Deficient Head and Neck Squamous Cell Carcinoma

In head and neck squamous cell carcinoma (HNSCC), a significant proportion of tumors have inactivating mutations in the histone methyltransferase NSD1. In these tumors, NSD1 inactivation is shown to be a driver of T cell exclusion from the tumor microenvironment (TME). Here, we demonstrated that NSD1 inactivation results in lower levels of H3K36 di-methylation and higher levels of H3K27 tri-methylation, the latter being a known repressive histone mark enriched on the promoters of key T cell chemokines CXCL9 and CXCL10. HNSCC with NSD1 mutations had lower levels of these chemokines and lacked responses to PD-1 immune checkpoint blockade. Inhibition of KDM2A, the primary lysine demethylase that is selective for H3K36, reversed the altered histone marks induced by NSD1 loss and restored T cell infiltration into the TME. Importantly, KDM2A suppression decreased growth of NSD1-deficient tumors in immunocompetent, but not in immunodeficient, mice. Together, these data indicate that KDM2A is an immunotherapeutic target for overcoming immune exclusion in HNSCC.

Because H3K27me3 is enriched on the repressed promoters of the key T cell recruiting chemokines CXCL9 an CXCL10, here, we test the hypothesis that loss of NSD1 function—and the subsequent effects on H3K36 and H3K27 methylation states—impacts CXCL9 and CXCL10 expression in the tumor microenvironment. Importantly, our studies demonstrate the potential of targeting the opposing H3K36me2-specific lysine demethylase, KDM2A, as a therapeutic strategy to reverse the effects of NSD1 inactivation in HNSCC and enhance T cell infiltration.

### Materials and Methods

Cell lines. The FaDu human HNSCC cell line was obtained from ATCC; early passage cells were used. The MOC1 cell line was provided by Dr. Ravindra Uppaluri, who developed the cell line from murine oral squamous cell carcinomas induced by topical 7,12-dimethylbenz(a) anthracene (DMBA) administration; early passage cells were used. Cells were cultured in complete DMEM/F12 medium containing 10% fetal bovine serum (FBS), 1% Non-Essential Amino Acid (NEAA) and 1% penicillin and streptomycin. Cells were maintained at 37 deg C in a humidified atmosphere containing 5% CO₂. The 293GP cell line (RRID:CVCL_E072) was a kind gift from Dr. Crystal Mackall (Department of Pediatrics—Hematology & Oncology, Stanford University). The Lenti-X 293T cell line was purchased from Takara Bio USA, Inc (RRID:CVCL_0063). Cells were cultured in complete DMEM medium containing 10% FBS, 1% penicillin and streptomycin. Cells were maintained at 37 deg C in a humidified atmosphere containing 5% CO₂.

T cells. Our use of human donor blood was approved by the Institutional Review Board at Stanford University. Leukoreduction system (LRS) chambers were obtained from the Stanford Blood Center. T cells were enriched using the RosetteSep™ Human T Cell Enrichment Cocktail (STEMCELL Technologies) followed by centrifugation on Ficoll-Paque™ Premium (GE Healthcare). T cells were cultured in complete RPMI/1640 medium containing 10% FBS, 1% penicillin and streptomycin, 100 U/mL IL-2 and 1% HEPES at 37 deg C in a humidified atmosphere containing 5% CO₂. T cells were activated by anti-CD3 and anti-CD28 antibody (Biolegend) according to the manufacturer's instructions 3 days before use. Plates were pre-coated with 50 μL of 10 μg/mL anti-CD3 and anti-CD28 antibody in each microwell of the 96-well plate at 4 deg C overnight. The wells were washed, and 200 μL of 1-2×10⁶/mL T cells were added to each well. The cells were incubated at 37 deg C in a humidified atmosphere containing 5% CO₂ for 3 days before use.

Lentiviral production and transduction. The Lenti-X 293T cell line was used to package and produce the lentiviral particles. Briefly, the cells were transfected with the packaging plasmid pCMV-dR8.2 (RRID:Addgene_8455), the envelope plasmid pCMV-VSV-G (RRID:Addgene_8454), and the transfer plasmids containing the shRNA or the transgene, using Lipofectamine® 2000 (ThermoFisher) according to the manufacturer's instructions. Supernatants were collected 48 to 72 hours post-transfection and used to transduce the target cells in 6-well plates at a concentration of 3×10⁵ cells per well with 8 μg/mL polybrene. The medium was changed 24 hours later.

Retroviral production and transduction. The 293GP cell line was used to produce the retroviral particles for CAR transduction. The 293GP cells were transfected with the envelope plasmid RD114 and the plasmids encoding CD19 CAR (a kind gift from Dr. Crystal Mackall), using Lipofectamine® 2000 according to the manufacturer's instructions. The supernatants were collected 48 to 72 hours post-transfection and then stored at −80 deg C. Retroviral supernatants were diluted 1:1 in T cell culture media and applied to non-treated 6-well plates (Corning) coated with retronectin (Takara). Plates were centrifuged at 3200 rpm for 2-3 hours at 32 deg C. Vector-containing supernatants were removed, and activated T cells (1×10⁶ cells/well) were added to the plates. The plates were centrifuged at 1000 g for 45 min at 32 deg C and then incubated at 37 deg C.

Mice. B6.129S7-Rag1^{tm1Mom}/J mice (Rag1 KO mice) (RRID:IMSR_JAX:002216) and C57BL/6J (B6 mice) (RRID:IMSR_JAX:000664) were obtained from Jackson Laboratory. The mice were housed in laminar flow cabinets under specific pathogen-free conditions and fed ad libitum. All procedures were performed in accordance with protocols approved by the Administrative Panel on Laboratory Animal Care at Stanford University. For in vivo experiments with murine HNSCC cell lines, 2×10⁶ cells were injected subcutaneously in the flanks of the Rag1 KO mice or wild type B6 mice. Tumor volume (mm³) was determined by caliper measurements performed every two to three days and calculated by using the following formula: volume=length×width²×0.5. The tumors were harvested and processed for TIL analysis.

Tumor dissociation and tumor-infiltrating lymphocyte analysis. Tumor tissue was digested as follows. The tissue was minced, transferred into gentleMACS™ C-tubes with digestion solution (DMEM-F12+1% FBS, 1% Pen strip, 25 mM HEPES and 10% collagenase/Hyaluronidase), and dissociated. The dissociated tissue was incubated while rotating at 37 deg C for 1 hour. After incubation, the suspension was filtered through a 40 μM filter. If the cell pellet was heavily contaminated with red blood cells, a brief ACK lysis was performed. The cells were washed and stained with antibodies to CD3 and CD45 (Biolegend). Data were acquired on a BD LSRFortessaor BD FACSAria II. Events collected were analyzed using FlowJo Version 10.5.0 software (RRID:SCR_008520).

Immunofluorescence (IF) staining and microscopy of tumor tissue. OCT-embedded tissue sample sections (3-5 μm) were dried for 30 minutes at room temperature and then in ice-cold acetone for 5-10 min. Washed sections were then permeabilized in 0.1% Triton X-100, blocked in 5% BSA for 60 min at room temperature. The slides were then stained with anti-CD3 antibody (Biolegend), anti-CXCL9 antibody (Invitrogen), or anti-CXCL10 antibody (Bioss) at 4 deg C overnight. Subsequently, the slides were incubated at room temperature for 1 hour with a secondary antibody (Invitrogen), followed by DAPI staining for 20 minutes. The samples were mounted on glass slides and analyzed by LSM700 confocal microscopy (Zeiss).

Quantitative RT-PCR. RNA was extracted with the RNeasy Mini Kit (Qiagen), and cDNA synthesis was performed using Maxima First Strand cDNA Synthesis Kit (Thermo Fisher Scientific) according to the manufacturer's protocol. The relative gene expression was analyzed using the Luminaris Color Probe High ROX qPCR Master Mix (Thermo). Relative gene expression was obtained by the ΔΔCt method (20) after normalization to HPRT1.

Western blot. Histone protein was extracted with the Histone Extraction Kit (Abcam), according to the manufacturer's protocol. The protein samples were denatured and separated by Novex™ WedgeWell™ 4-12% Tris-Glycine Mini Gels (Thermo Fisher Scientific) and transferred onto 0.2 μm PVDF Pre-cut Blotting Membranes (Thermo Fisher Scientific). After blocking with 5% non-fat milk at room temperature for 2 hours, the membranes were incubated with primary antibodies to H3K36me2 or H3K27me3 or total H3 at 4 deg C overnight. Subsequently, the membranes were incubated with secondary antibodies for 1 hour at room temperature. Finally, protein signals were detected by Pierce™ ECL Western Blotting Substrate (Thermo Fisher Scientific) and visualized after exposure to the Hyperfilm™ ECL™ (GE Healthcare). The relative optical density ratio was calculated with the Image J software (RRID:SCR_003070) by comparison to H3.

Spheroid T cellinfiltration assay. CD19 (truncated) expressing FaDu cells (7×10³) were resuspended in 1 mL of liquefied Matrigel on ice. Aliquots of 30 μL of resuspended cells in Matrigel were plated as droplets in the center of wells of a 24-well plate. Plates were incubated at 37 deg C in 5% CO2 for 15 minutes to solidify Matrigel domes. Domes were overlaid with 1 mL of EN medium and incubated at 37 deg C in a 5% CO2 humidified incubator. EN medium consisted of DMEM/F12 medium containing 10 mM Nicotinomide, 1 mM N-acethylcysteine, 1×B-27™ Supplement, 1× Antibiotic-Antimycoti, 50 ng/mL EGF, and 100 ng/mL Noggin. Culture medium was changed twice a week. After 2 weeks, 2×10⁵ CD19 CAR T cells were added to each well. For the KDM2A inhibitor treated group, 1.5 μM daminozide was added to the medium the day before adding the T cells. After 3 days of co-culture, the spheroid Matrigel domes were harvested and analyzed by microscopy. Domes were fixed with 4% paraformaldehyde for 30 minutes at room temperature. The domes were washed with PBS three times, and the organoids were then incubated in 0.5% Triton X-100+5% BSA in PBS for 2 hours at room temperature. The permeabilized spheroids were then incubated with antibodies at 4 deg C overnight, washed, and then incubated with DAPI for 20 mins. The stained samples were mounted on glass slides and analyzed by LSM700 confocal microscopy (Zeiss).

Enzyme-linked immunosorbent assay. CXCL9 and CXCL10 protein levels in the conditioned culture medium were measured by enzyme-linked immunosorbent assay (ELISA), using the Human CXCL9 (MIG) Mini ABTS ELISA Development Kit (Peprotech) and the Human CXCL10 (IP-10) ELISA MAX™(BioLegend), following the manufacturer's instructions. Briefly, plates were pre-coated with anti-CXCL9 or anti-CXCL10 antibody. Standards and cell culture supernatants were diluted in sample diluent buffer and incubated for 2 hours at room temperature. Detection antibodies were diluted in antibody diluent buffer and incubated for 2 hours at room temperature. After washing steps, HRP-conjugate was diluted and incubated for 30 minutes at room temperature. After washing steps, ABTS liquid substrate was added to each well, and absorbance was measured at a wavelength of 405 nm with correction wavelength of 650 nm using a microplate (ELISA) reader (SpectraMax M3, Molecular Devices). An eight-point standard curve was used to calculate the concentration (pg/mL) of CXCL9 and CXCL10 in the samples.

TCGA and cBioPortal. The cBioPortal (RRID:SCR_014555) was used to analyze the HPV-negative Head and Neck Squamous Cell Carcinoma (TCGA, PanCancer Atlas) dataset.

CXCL9 and CXCL10 in human HNSCC datasets. RNAseq data from a neoadjuvant pembrolizumab HNSCC cohort was examined for CXCL9 and CXCL10 transcripts. The scRNA-Seq dataset, which was generated using Smart-Seq2 technology as part of a study reported by (24), was accessed from Gene Expression Omnibus (RRID:SCR_005012) (Accession number: GSE103322) as a preprocessed series matrix file. The dataset was subsequently analyzed using Seurat (25): The series matrix was loaded into a Seurat object and was filtered to exclude poor quality or dying cells by removing cells with a mitochondrial genome fraction of 0.4 or greater as well as those with a unique feature count of fewer than 200 genes. Potential doublets were excluded by removing cells with a unique feature count of greater than 4000. Integration was then performed by splitting the dataset into separate Seurat objects, with each object containing all the cells that derived from one sample. Gene expression counts for each cell were normalized using regularized negative binomial regression, and variable genes (N=2000) were found for each sample using the ‘vst’ method. Samples were then integrated into a single gene expression object by finding integration anchors using the ‘FindIntegrationAnchors’ and ‘IntegrateData’ commands. The combined genes were then scaled and centered using linear models. This integration approach resulted in clustering of cells by cell type and cell cycle stage rather than by sample. Cell type labels that were previously assigned by Puram et al. were accessed from the GEO meta-data. Expression of CXCL9 and CXCL10 was analyzed within samples that had been profiled using whole exome sequencing, such that the NSD1 mutation status was known. NSD1 somatic mutation calls were accessed from the Puram et al report. For each gene (CXCL9 and CXCL10), Fisher's extract test was used to test for differences the number of malignant cells that expressed the gene (i.e., had a normalized count value greater than zero) between NSD1 mutated and NSD1 wild type HNSCC samples. For primary and metastatic tumors independently, comparisons were made between malignant cells of the NSD1 HNSCC versus malignant cells of each NSD1 wild-type HNSCC separately.

ChIP-seq analysis. The H3K36me2 and H3K27me3 ChIP-seq data were downloaded from GSE149670. ChIP-seq clean reads were aligned to the human genome (GRCh38) using Bowtie2 (RRID:SCR_016368). Then, aligned reads were filtered to remove PCR duplicates and reads from chromosomes 1-21, X and Y are retained. ChIP-seq signal profiles were converted to BigWig tracks using deeptools (28), then visualized by Integrated Genome Viewer (IGV).

Statistical analysis. Data were expressed as mean±standard error of mean (SEM) and analyzed by t-test or one way ANOVA. Statistical analysis was performed with GraphPad Prism software (RRID:SCR_002798). Values of p<0.05 were considered to be statistically significant.

### Results

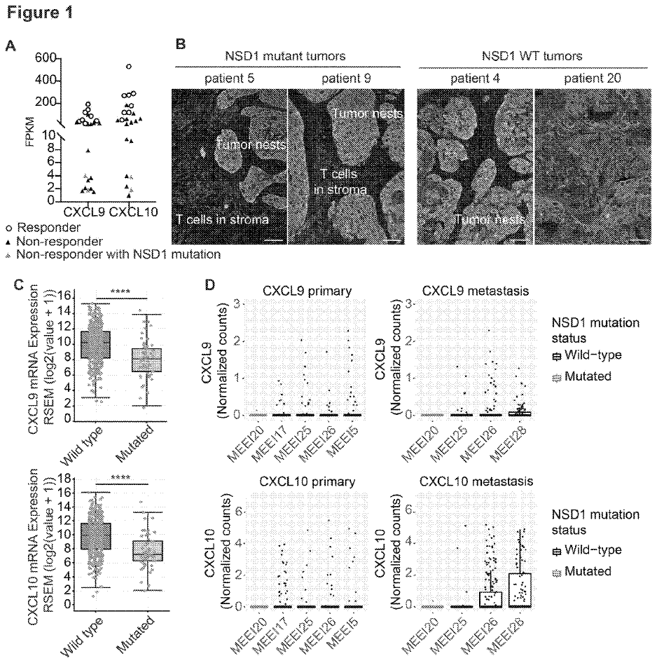

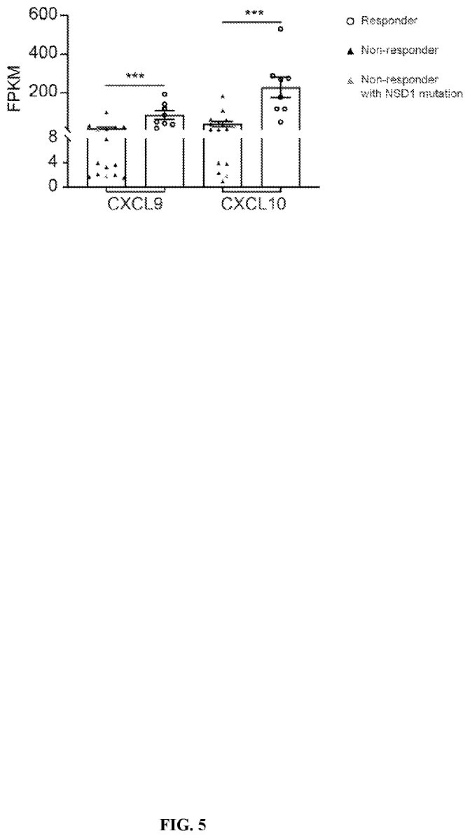

Inactivation of NSD1 results in downregulated expression of CXCL9 and CXCL10 in HNSCC. Analysis of data from a multicenter, phase II, window-of-opportunity trial (NCT02296684), in which neoadjuvant pembrolizumab was administered to patients with locally advanced, HPV-unrelated HNSCC two to three weeks prior to definitive surgical resection, revealed a significant correlation between pathologic tissue response and expression of the key T cell-recruiting chemokines CXCL9 and CXCL10 (FIG. 1A, FIG. 5). None of the tumors with low CXCL9 or CXCL10 expression exhibited response to pembrolizumab. Two of the patients in the study had tumors with inactivating mutations in NSD1. Both were non-responders and were among those with the lowest expression of CXCL9 and CXCL10. Interestingly, histologic examination of the NSD1-mutant tumors demonstrated a microenvironment that was devoid of T cells at baseline (FIG. 1B). Notably, the T cells appeared to be excluded from the tumor microenvironment and confined to the stromal compartment between the tumor cell nests.

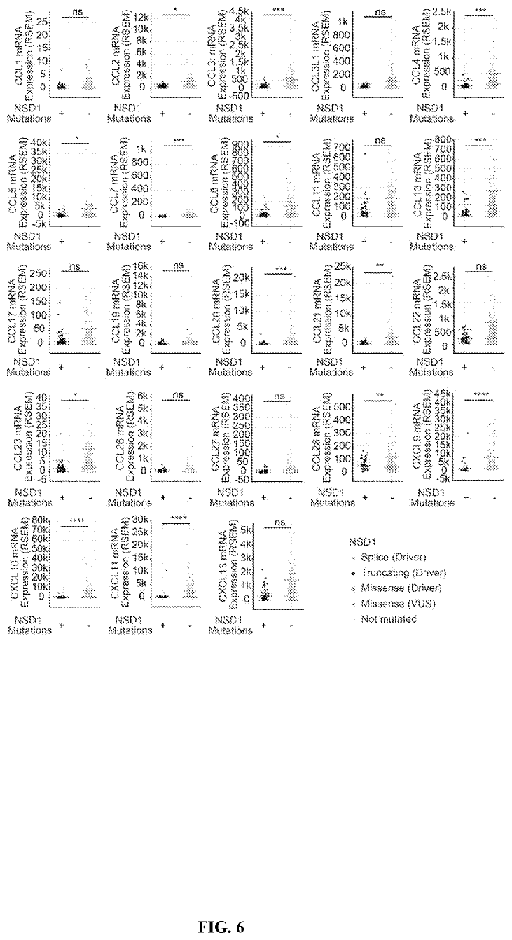

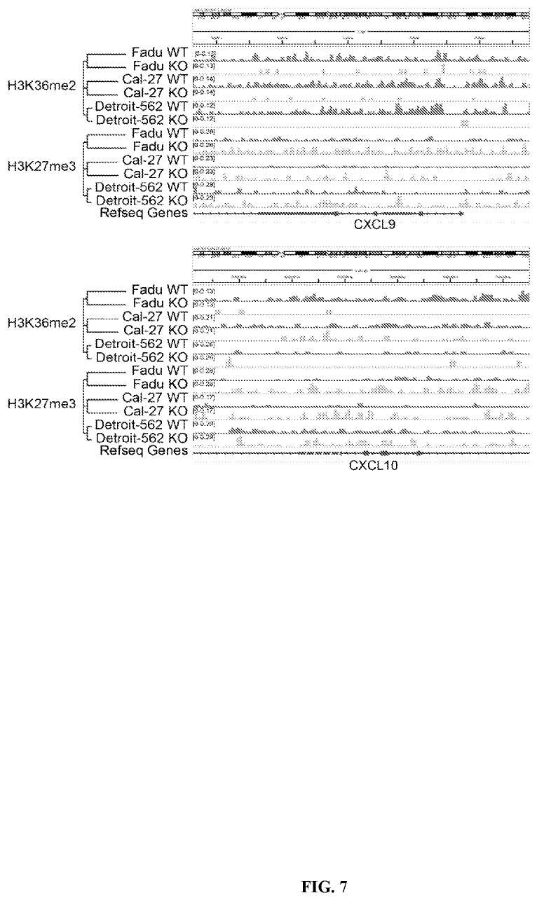

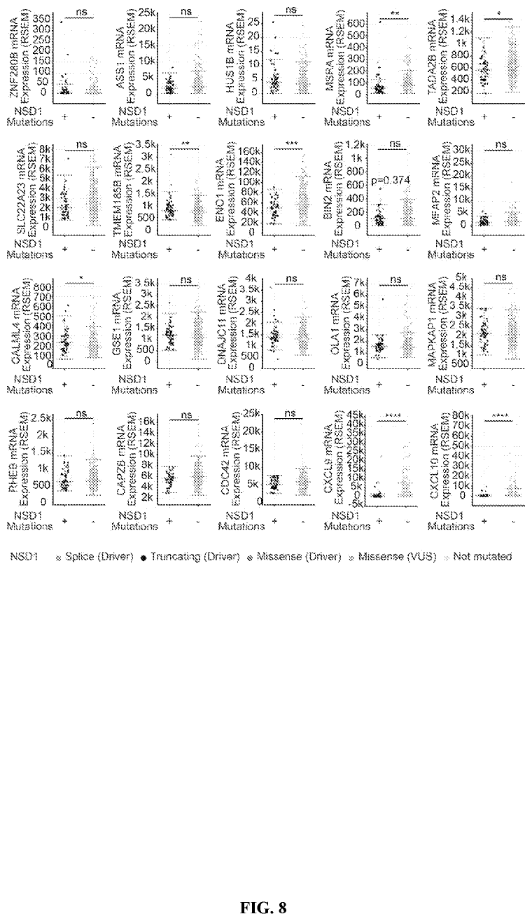

To investigate further the relationship between NSD1 mutations and CXCL9/CXCL10 expression, we examined previously reported human HNSCC datasets. In The Cancer Genome Atlas (TCGA), HNSCC tumors with NSD1 mutations had significantly lower expression of CXCL9/CXCL10 (FIG. 1C). In fact, of all T cell related chemokines/cytokines that had differences in expression, CXCL9 and CXCL10 had the most significantly altered expression levels in an NSD1 mutant background (FIG. 6) and have an important role in the recruitment of T cells into the tumor microenvironment. Similarly, analysis of CXCL9 and CXCL10 expression in a previously published HNSCC single cell RNA-sequencing (scRNA-seq) dataset also revealed essentially absent expression of these chemokines in the context of NSD1 mutations (FIG. 1D). This dataset included scRNA-seq profiles for a primary and a metastatic (lymph node metastasis) tumor of an HPV-negative patient that had a somatic point mutation (nonsense) in NSD1 (detected by whole exome sequencing), as well as primary (N=4) and metastatic (N=3) tumors of NSD1 wild-type HNSCCs (i.e., tumors in which NSD1 mutations were not detected). This revealed that CXCL9 and CXCL10 were expressed (i.e., detected) within a subset of malignant cells of each primary or metastatic NSD1 wild-type HNSCC, but were undetected or detected in only one malignant cell of the NSD1 mutated primary and metastatic HNSCCs. Indeed, for both primary and metastatic HNSCCs, the frequency of cells with detectable CXCL9 or CXCL10 expression was significantly lower within malignant cells of the NSD1 mutated tumor compared with malignant cells of each NSD1 wild-type counterpart. Finally, because NSD1-mediated catalysis of H3K36 di-methylation directly antagonizes H3K27 tri-methylation by PRC2, we hypothesized that NSD1 inactivation would correlate with enrichment of H3K27me3 on CXCL9 and CXCL10 loci, a known repressive mark for these genes, and indeed, analysis of published ChIP-seq data from HNSCC cell lines revealed an increase in H3K27me3 and a reduction in H3K36me2 on CXCL9 and CXCL10 loci when NSD1 was knocked out (FIG. 7). Further, of known H3K27me3-regulated genes, CXCL9 and CXCL10 were among those with the most significantly altered expression in the NSD1 mutant subset of HNSCC tumors in the TCGA (FIG. 8). Together, these data indicate a downstream role of NSD1 inactivation on CXCL9 and CXCL10 expression and point to disruption of the NSD1-CXCL9/CXCL10 relationship being associated with resistance to immune checkpoint blockade.

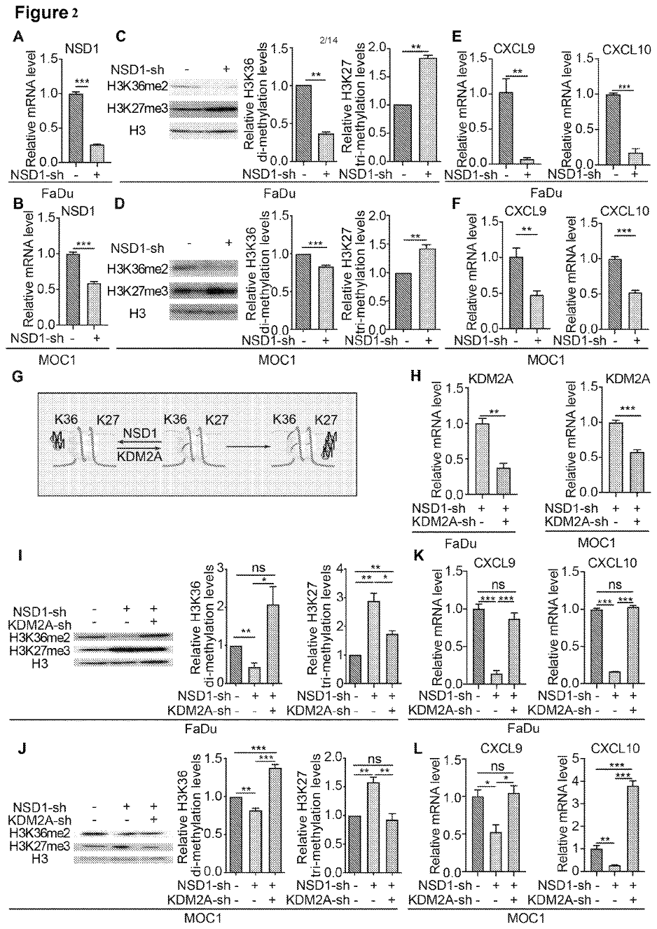

The effect of NSD1 inactivation on CXCL9 and CXCL10 expression can be reversed by inactivation of KDM2A. We next investigated the effects of NSD1 inactivation on H3K36 and H3K27 methylation and chemokine expression. Lentiviral transduction of NSD1 shRNA in human and mouse HNSCC cells resulted in a decrease in H3K36me2 and an increase in H3K27me3, as well as a concomitant decrease in CXCL9 and CXCL10 expression (FIGS. 2A-F), in line with what we observed in the human HNSCC datasets (FIG. 1).

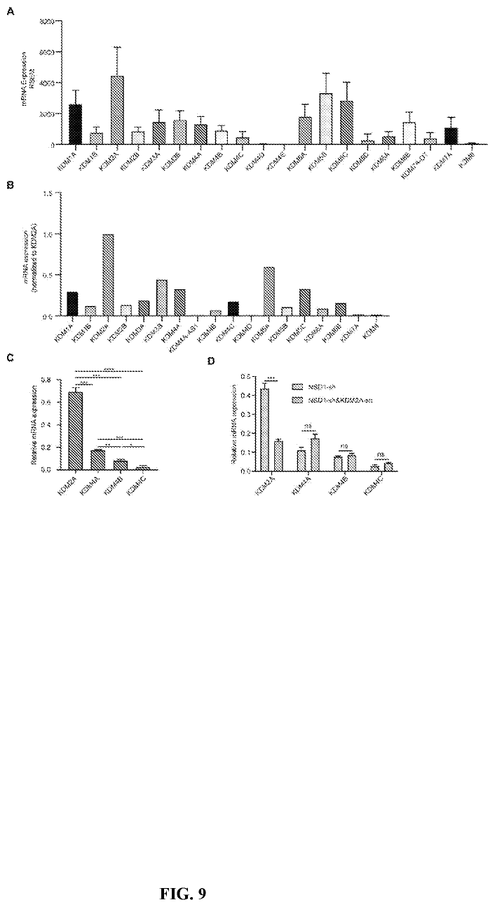

The JHDM1/KDM2 and JHDM3/JMJD2/KDM4 family comprise the two lysine demethylase families with activity on methylated H3K36. Notably, the JHDM1/KDM2 family, which consists of two human members, KDM2A and KDM2B, is selective for lower methylation states of H3K36 (31). In HNSCC tumors, KDM2A is the most highly expressed H3K36-active lysine demethylase gene (FIG. 9A-B). We independently observed the relatively high expression of KDM2A in our cell lines (FIG. 9C) and thus, hypothesized that in the context of NSD1 inactivation, the targeted inhibition of KDM2A expression might be able to restore methylation of H3K36 (via other secondary methyl transferases other than NSD1). Because tri-methylation of H3K27 by PRC2 is directly antagonized by H3K36 tri-methylation, we also hypothesized that inhibition of KDM2A would indirectly lead to decreased levels of H3K27me3 (FIG. 2G).

As previously observed, inhibited expression of NSD1 resulted in decreased H3K36me2 levels and increased H3K27me3 levels (FIG. 2I-J; Supp Fig. R1). Strikingly, the concurrent knockdown of KDM2A in the NSD1 knockdown cells was sufficient to restore (and even exceed) H3K36me2 levels compared to the parental controls. Similarly, the levels of H3K27me3 levels were reduced closer to baseline control levels with the concomitant KDM2A knockdown. Thus, KDM2A targeting was sufficient to reverse the histone methylation effects of NSD1 inhibition in these cells.

These effects on H3K36 and H3K27 methylation states by KDM2A inhibition also had significant impact on the expression of CXCL9 and CXCL10. KDM2A knockdown resulted in the increased expression of CXCL9 and CXCL10 that was equal to or greater than the expression seen in the parental cells (FIGS. 2K and 2L)—indicating that the indirect effects of targeting KDM2A on the methylation state of H3K27 has physiologic relevance.

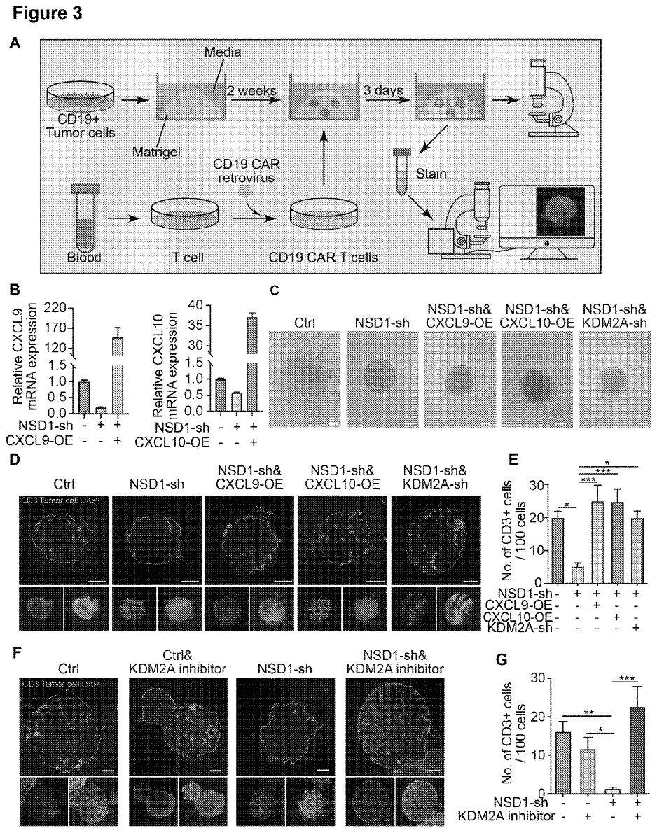

Inhibition of KDM2A enhances T cell infiltration into the tumor microenvironment of HNSCC. As mentioned, NSD1 inactivating mutations in HNSCC have a strong correlation with an immune cell-deficient tumor microenvironment. Given our observations of NSD1 inactivation on CXCL9 and CXCL10 expression (FIG. 1-2), we investigated the role of these chemokines on T cell recruitment in the context of altered NSD1 and KDM2A activity. To do this, we utilized in vitro cultured 3-dimensional tumor spheroids grown from HNSCC cell lines that could be manipulated to express chemokines and/or shRNA (FIG. 3A-B). Co-culture of these spheroids with human donor-derived T cells enabled the assessment of T cell infiltration into the tumor microenvironment. Because these were not tumor antigen-specific T cells, we transduced the T cells to express a CD19-specific chimeric antigen receptor (CD19-CAR) and the tumor cells to express a truncated surface CD19 molecule that lacked the intracellular portion. In this manner, the CD19-CAR provided T cell specificity for the tumor cells, and we were able to use this system to assess how tumor cell-intrinsic mechanisms influence T cell infiltration.

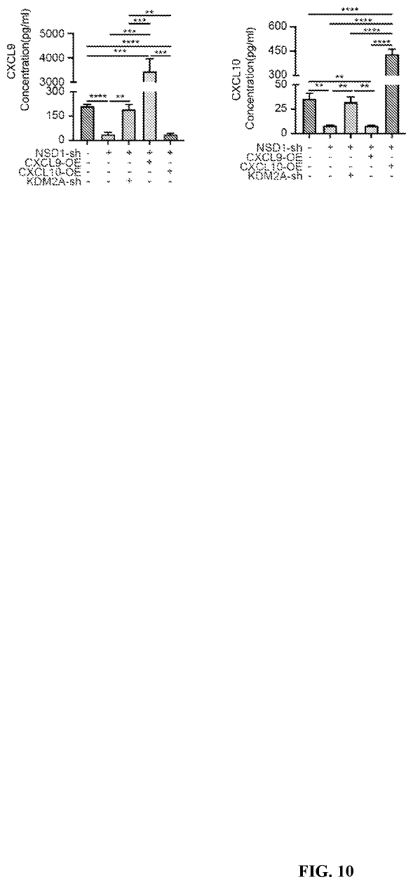

The CD19-expressing tumor cells were transduced to express shRNA targeting either NSD1 or KDM2A and/or transduced to express either CXCL9 or CXCL10 prior to forming tumor spheroids. The expression of the chemokines was confirmed by qRT-PCR (FIG. 3B) and ELISA (FIG. 10). The spheroids grew in a similar fashion compared to each other and compared to control transduced cells; however, the spheroids expressing the shRNA targeting NSD1 had a distinctly sharper border and had fewer T cells aggregating near them when in co-culture (FIG. 3C). To assess the ability of the T cells to infiltrate the microenvironment of the spheroids, we analyzed co-cultured spheroids by confocal immunofluorescence microscopy using labeled anti-CD3 antibody (FIG. 3D). The number of T cells in each spheroid was quantified relative to the number of total cells (FIG. 3E). We observed significantly fewer infiltrating T cells in the NSD1 knockdown spheroids compared to control spheroids. Notably, forced expression of CXCL9 or CXCL10 by the tumor cells was sufficient to restore the number of infiltrating T cells in the context of NSD1 knockdown.

This forced expression of CXCL9 and CXCL10 was supraphysiologic, and thus, other mechanisms may be involved in T cell exclusion in NSD1-mutant tumors besides the suppressed expression of CXCL9/10. Nevertheless, inhibition of KDM2A expression restored T cell infiltration in the NSD1 knockdown tumor spheroids (FIG. 3D-E), consistent with the ability of KDM2A inhibition to rescue CXCL9 and CXCL10 expression in NSD1 knockdown tumor cells (FIGS. 2K and 2L). An orthogonal approach of using a KDM2A small molecule inhibitor (daminozide) also increased the number of infiltrating T cells into tumor spheroids, similar to what was observed with the shRNA inhibition of KDM2A expression (FIG. 3F-G). Thus, targeting KDM2A in NSD1-inactivated tumors can rescue T cell infiltration into the microenvironment.

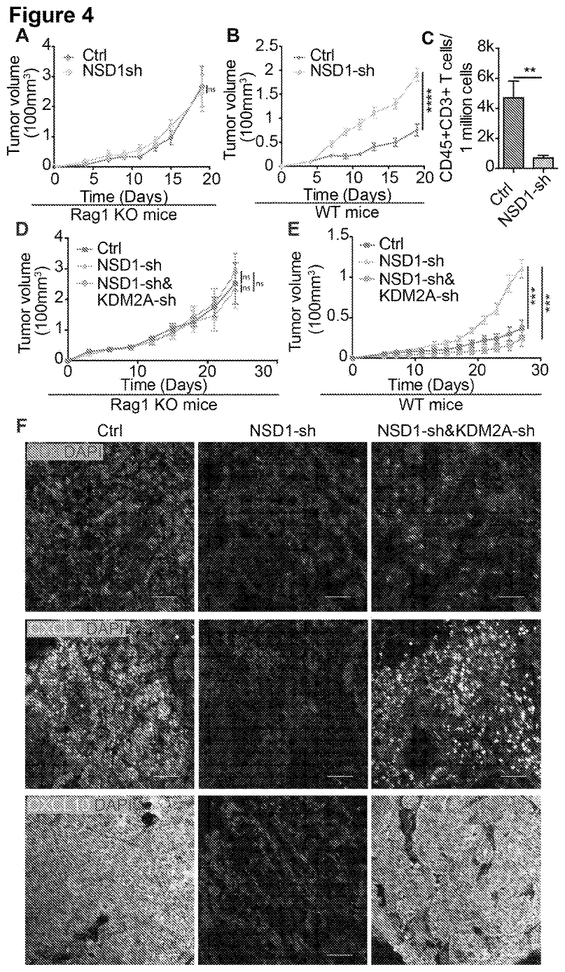

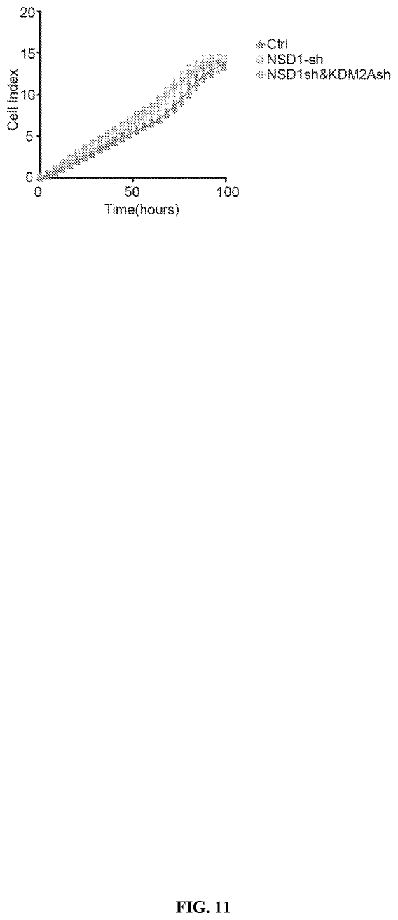

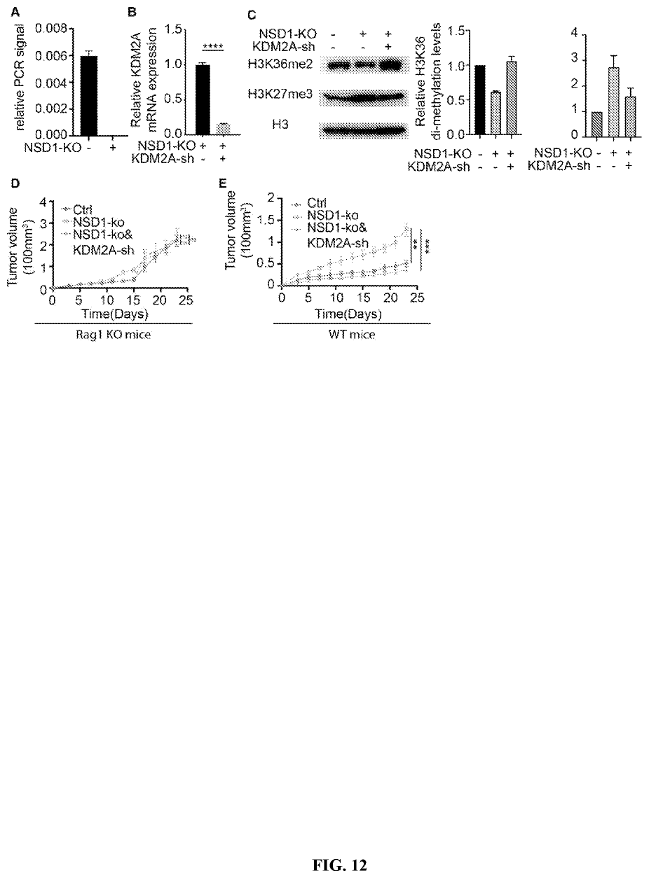

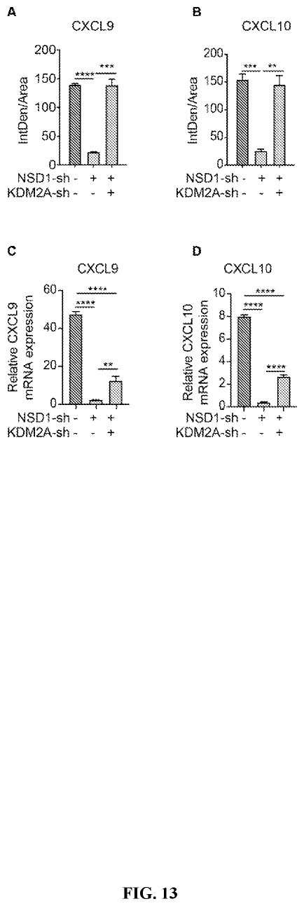

KDM2A inhibition restores T cell infiltration into immune cell-depleted NSD1-inactivated tumors in vivo. As mentioned, inhibition of NSD1 and/or KDM2A expression did not have any apparent effect on tumor spheroid growth in vitro (FIG. 3C). We also confirmed that expression of shRNA targeting NSD1 and KDM2A did not affect tumor cell proliferation kinetics in vitro (FIG. 11). Similarly, inhibition of NSD1 and KDM2A in a C57BL/6 murine oral cancer model (MOC1) did not have any effect on tumor growth kinetics in vivo when assessed in syngeneic C57BL/6 Rag1-deficient (Rag1 KO) mice, which lack T and B cells (FIG. 4A; FIG. 12). However, interestingly, when assessed in immunocompetent wild-type (WT) C57BL/6 mice, NSD1 inhibition or inactivation in MOC1 cells resulted in significantly greater tumor volumes compared to control MOC1 cells (FIG. 4B; FIG. 12). The concomitant inhibition of KDM2A expression in the MOC1-NSD1 shRNA cells inhibited the tumor volumes back to levels of the control MOC1 cells in WT mice (FIG. 4E; FIG. 12) but not in Rag1 KO mice (FIG. 4D; FIG. 12), indicating that tumor inhibition was immune mediated. Consistent with our tumor spheroid data (FIG. 3D-G), we found that there were very few CD45⁺ CD3⁺ T cells in the MOC1 tumors expressing shRNA targeting NSD1 in WT mice, as assessed by flow cytometry of suspended cells from dissociated tumors and by immunofluorescence of tissue section (FIGS. 4C and 4F). Further, there was little to no expression of CXCL9 and CXCL10 seen in these tumors (FIG. 4F). However, the concomitant expression of shRNA targeting KDM2A was sufficient to restore both the chemokine expression and CD3⁺ T cell infiltration into the tumor microenvironment of NSD1 knockdown MOC1 cells grown in WT mice (FIG. 4F, FIG. 13). Thus, the inhibition of KDM2A provides a powerful strategy for improving T cell infiltration into HNSCC tumors devoid of immune cells and offers a potential immunotherapeutic approach to enhance responses to immune checkpoint blockade.

Successful anti-tumor immune responses following PD-1/PD-L1 checkpoint blockade require recruitment and clonal proliferation of tumor-specific T cells present in the tumor microenvironment (TME). Tumor expression of the interferon (IFN) γ-inducible chemokines CXCL9 (also known as monokine induced by gamma interferon, or MIG) and CXCL10 (also known as interferon γ-induced protein 10, or IP-10) is correlated with the presence of tumor infiltrating CD8+ T cells and the activation of Th1 type immunity within the TME. As such, these chemokines are considered critical for robust responses to immune checkpoint inhibitors (e.g. anti-PD-1 and anti-CTLA-4 antibodies) and have been associated with improved patient outcomes.

A subset of HNSCC with mutations in NSD1 has an immune-cold TME, and we previously demonstrated that inactivation of this H3K36-specific histone methyltransferase can induce a TME phenotype that is resistant to T cell infiltration. Here, we report that the expression of CXCL9 and CXCL10 is repressed in the context of NSD1 inactivation and that targeting of KDM2A, a lysine demethylase specific for H3K36me2, leads to restored expression of these chemokines and subsequent recruitment of immune cells into the TME. The repression of CXCL9 and CXCL10 is likely due to the increased levels of H3K27me3 observed when NSD1 is inactivated.

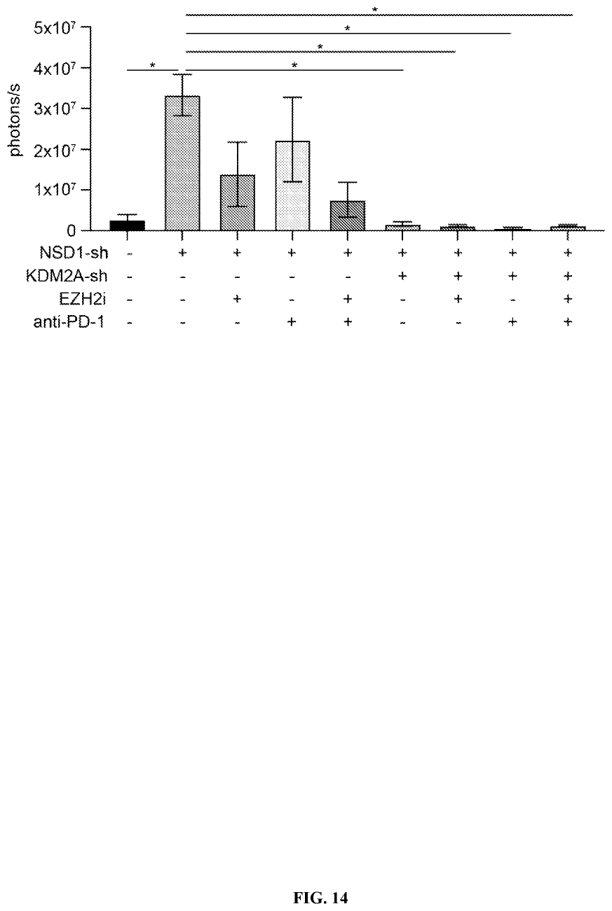

The ability to inhibit H3K27 tri-methylation indirectly by targeting KDM2A has implications for cancer therapeutic approaches. EZH2 overexpression and H3K27me3 dysregulation are observed in many cancers, and EZH2's catalytic (methyltransferase) and noncatalytic roles have been shown to contribute to tumor development and progression. In HNSCC, H3K27 tri-methylation resulting from EZH2 overexpression is negatively correlated with MHC I expression on the tumor cells and response to PD-1 checkpoint blockade. This and other preclinical immune studies have led to the proposal of combining EZH2 inhibition with immunotherapy. Over the years, significant effort has been directed toward the development of approaches to target EZH2, and several small molecule inhibitors of EZH2 are now in early clinical trials. However, EZH2 is also critical for CD8⁺ effector T cell survival and antitumor activity and is important for Th1 and Th2 differentiation from naïve CD4 T cells. Thus, some concern has been raised regarding the possibility that T cell dysfunction may be observed with global EZH2 inhibition, and thus, there is rationale to explore alternative ways to reverse the repressive effects of H3K27me3 on key promoter regions. In the NSD1-inhibited MOC1 mouse model, we observed that EZH2 inhibition does reduce tumor growth in WT mice but not to the extent that KDM2A inhibition appeared to achieve (FIG. 14). There is evidence that NSD1 inactivation results in resistance to EZH2 inhibition and that KDM2A inhibition can overcome this resistance.

Here, our study demonstrates that inhibition of KDM2A induces the increased methylation of H3K36 and decreased methylation of H3K27, resulting in both the increased expression of CXCL9/CXCL10 and the increased infiltration of T cells into the TME. The co-inactivation of KDM2A was able to reverse these effects of NSD1 inactivation on T cell infiltration, and thus, these data introduce KDM2A as a novel target for immunotherapy. Since response rates to immune checkpoint blockade correlate with the extent of immune cell infiltration in the TME, KDM2A inhibition can enhance clinical response to anti-PD-1 therapy.

### REFERENCES

Johnson D E, Burtness B, Leemans C R, Lui V W Y, Bauman J E, Grandis J R. Head and neck squamous cell carcinoma. Nat Rev Dis Primers [Internet]. 2020; 6:92.
Zolkind P, Uppaluri R. Checkpoint immunotherapy in head and neck cancers. Cancer and Metastasis Reviews [Internet]. Springer US; 2017 [cited 2018 Dec. 29]; 36:475-89.
Vaddepally R K, Kharel P, Pandey R, Garje R, Chandra A B. Review of Indications of FDA-Approved Immune Checkpoint Inhibitors per NCCN Guidelines with the Level of Evidence. Cancers (Basel) [Internet]. MDPI; 2020; 12:738.
Ferris R L, Blumenschein G J, Fayette J, Guigay J, Colevas A D, Licitra L, et al. Nivolumab for Recurrent Squamous-Cell Carcinoma of the Head and Neck. N Engl J Med. 2016; 375:1856-67.
Burtness B, Harrington K J, Greil R, Soulières D, Tahara M, de Castro G J, et al. Pembrolizumab alone or with chemotherapy versus cetuximab with chemotherapy for recurrent or metastatic squamous cell carcinoma of the head and neck (KEYNOTE-048): a randomised, open-label, phase 3 study. Lancet. England; 2019; 394:1915-28.
Cohen E E W, Bell R B, Bifulco C B, Burtness B, Gillison M L, Harrington K J, et al. The Society for Immunotherapy of Cancer consensus statement on immunotherapy for the treatment of squamous cell carcinoma of the head and neck (HNSCC). J Immunother Cancer. 2019; 7:184.
Oliva M, Spreafico A, Taberna M, Alemany L, Coburn B, Mesia R, et al. Immune biomarkers of response to immune-checkpoint inhibitors in head and neck squamous cell carcinoma. Ann Oncol [Internet]. Oxford University Press; 2019; 30:57-67.
Bonaventura P, Shekarian T, Alcazer V, Valladeau-Guilemond J, Valsesia-Wittmann S, Amigorena S, et al. Cold Tumors: A Therapeutic Challenge for Immunotherapy. Front Immunol. 2019; 10:168.
Daud Al, Loo K, Pauli M L, Sanchez-Rodriguez R, Sandoval P M, Taravati K, et al. Tumor immune profiling predicts response to anti-PD-1 therapy in human melanoma. J Clin Invest [Internet]. American Society for Clinical Investigation; 2016 [cited 2018 Mar. 17]; 126:3447-52.
Brennan K, Shin J H, Tay J K, Prunello M, Gentles A J, Sunwoo J B, et al. NSD1 inactivation defines an immune cold, DNA hypomethylated subtype in squamous cell carcinoma. Sci Rep [Internet]. 2017 [cited 2018 Dec. 30]; 7:17064.
Pan C, Izreig S, Yarbrough W G, Issaeva N. NSD1 mutations by HPV status in head and neck cancer: differences in survival and response to DNA-damaging agents. Cancers Head Neck [Internet]. 2019; 4:3.
Brennan K, Koenig J L, Gentles A J, Sunwoo J B, Gevaert O. Identification of an atypical etiological head and neck squamous carcinoma subtype featuring the CpG island methylator phenotype. EBioMedicine [Internet]. 2017/03/01. Elsevier; 2017; 17:223-36.
Li Y, Goldberg E M, Chen X, Xu X, McGuire J T, Leuzzi G, et al. Histone methylation antagonism drives tumor immune evasion in squamous cell carcinomas. Mol Cell [Internet]. Elsevier; 2022; 82:3901-3918.e7.
Jaffe J D, Wang Y, Chan H M, Zhang J, Huether R, Kryukov G V, et al. Global chromatin profiling reveals NSD2 mutations in pediatric acute lymphoblastic leukemia. Nat Genet. 2013; 45:1386-91.
Streubel G, Watson A, Jammula S G, Scelfo A, Fitzpatrick D J, Oliviero G, et al. The H3K36me2 Methyltransferase Nsd1 Demarcates PRC2-Mediated H3K27me2 and H3K27me3 Domains in Embryonic Stem Cells. Mol Cell. United States; 2018; 70:371-379.e5.
Sengupta D, Zeng L, Li Y, Hausmann S, Ghosh D, Yuan G, et al. NSD2 dimethylation at H3K36 promotes lung adenocarcinoma pathogenesis. Mol Cell. 2021; 81:4481-4492.e9.
Yuan G, Flores N M, Hausmann S, Lofgren S M, Kharchenko V, Angulo-Ibanez M, et al. Elevated NSD3 histone methylation activity drives squamous cell lung cancer. Nature. 2021; 590:504-8.
Nagarsheth N, Peng D, Kryczek I, Wu K, Li W, Zhao E, et al. PRC2 Epigenetically Silences Th1-Type Chemokines to Suppress Effector T-Cell Trafficking in Colon Cancer. Cancer Res [Internet]. NIH Public Access; 2016 [cited 2018 Mar. 17]; 76:275-82.
Long A H, Haso W M, Shern J F, Wanhainen K M, Murgai M, Ingaramo M, et al. 4-1BB costimulation ameliorates T cell exhaustion induced by tonic signaling of chimeric antigen receptors. Nat Med [Internet]. 2015; 21:581-90.
Livak K J, Schmittgen T D. Analysis of Relative Gene Expression Data Using Real-Time Quantitative PCR and the 2-ΔΔCT Method. Methods [Internet]. 2001; 25:402-8.
Cerami E, Gao J, Dogrusoz U, Gross B E, Sumer S O, Aksoy B A, et al. The cBio cancer genomics portal: an open platform for exploring multidimensional cancer genomics data. Cancer Discov. 2012; 2:401-4.
Gao J, Aksoy B A, Dogrusoz U, Dresdner G, Gross B, Sumer S O, et al. Integrative analysis of complex cancer genomics and clinical profiles using the cBioPortal. Sci Signal. 2013; 6:pl1.
Uppaluri R, et al. Neoadjuvant and Adjuvant Pembrolizumab in Resectable Locally Advanced, Human Papillomavirus-Unrelated Head and Neck Cancer: A Multicenter, Phase II Trial. Clin Cancer Res. 2020; 26:5140-52.
Puram S V, Tirosh I, Parikh A S, Patel A P, Yizhak K, Gillespie S, et al. Single-Cell Transcriptomic Analysis of Primary and Metastatic Tumor Ecosystems in Head and Neck Cancer. Cell. 2017; 171:1611-1624.e24.
Hao Y, Hao S, Andersen-Nissen E, Mauck W M 3rd, Zheng S, Butler A, et al. Integrated analysis of multimodal single-cell data. Cell. 2021; 184:3573-3587.e29.
Farhangdoost N, Horth C, Hu B, Bareke E, Chen X, Li Y, et al. Chromatin dysregulation associated with NSD1 mutation in head and neck squamous cell carcinoma. Cell Rep. 2021; 34:108769.
Langmead B, Salzberg S L. Fast gapped-read alignment with Bowtie 2. Nat Methods [Internet]. 2012; 9:357-9.
Ramírez F, Ryan D P, Grüning B, Bhardwaj V, Kilpert F, Richter A S, et al. deepTools2: a next generation web server for deep-sequencing data analysis. Nucleic Acids Res. 2016; 44:W160-5.
Strazza M, Mor A. Consider the chemokines: a review of the interplay between chemokines and T cell subset function. Discov Med. United States; 2017; 24:31-9.
Cai Y, Zhang Y, Loh Y P, Tng J Q, Lim M C, Cao Z, et al. H3K27me3-rich genomic regions can function as silencers to repress gene expression via chromatin interactions. Nat Commun. 2021; 12:719.
Tsukada Y, Fang J, Erdjument-Bromage H, Warren M E, Borchers C H, Tempst P, et al. Histone demethylation by a family of JmjC domain-containing proteins. Nature. England; 2006; 439:811-6.
Majzner R G, Rietberg S P, Sotillo E, Dong R, Vachharajani V T, Labanieh L, et al. Tuning the Antigen Density Requirement for CAR T-cell Activity. Cancer Discov. 2020; 10:702-23.
Dangaj D, Bruand M, Grimm A J, Ronet C, Barras D, Duttagupta P A, et al. Cooperation between Constitutive and Inducible Chemokines Enables T Cell Engraftment and Immune Attack in Solid Tumors. Cancer Cell [Internet]. 2019; 35:885-900.e10.
Tokunaga R, Zhang W, Naseem M, Puccini A, Berger M D, Soni S, et al. CXCL9, CXCL10, CXCL11/CXCR3 axis for immune activation—A target for novel cancer therapy. Cancer Treat Rev [Internet]. 2017/11/26. 2018; 63:40-7.
Yuan S, Natesan R, Sanchez-Rivera F J, Li J, Bhanu N V, Yamazoe T, et al. Global Regulation of the Histone Mark H3K36me2 Underlies Epithelial Plasticity and Metastatic Progression. Cancer Discov [Internet]. 2020; 10:854-71.
Brown M A, Sims R J 3rd, Gottlieb P D, Tucker P W. Identification and characterization of Smyd2: a split SET/MYND domain-containing histone H3 lysine 36-specific methyltransferase that interacts with the Sin3 histone deacetylase complex. Mol Cancer. 2006; 5:26.
Ohtomo-Oda R, Komatsu S, Mori T, Sekine S, Hirajima S, Yoshimoto S, et al. SMYD2 overexpression is associated with tumor cell proliferation and a worse outcome in human papillomavirus-unrelated nonmultiple head and neck carcinomas. Hum Pathol. United States; 2016; 49:145-55.
Ezponda T, Licht J D. Molecular pathways: deregulation of histone h3 lysine 27 methylation in cancer-different paths, same destination. Clin Cancer Res. 2014; 20:5001-8.
Eich M-L, Athar M, Ferguson J E 3rd, Varambally S. EZH2-Targeted Therapies in Cancer: Hype or a Reality. Cancer Res. 2020; 80:5449-58.
Zhou L, Mudianto T, Ma X, Riley R, Uppaluri R. Targeting EZH2 Enhances Antigen Presentation, Antitumor Immunity, and Circumvents Anti-PD-1 Resistance in Head and Neck Cancer. Clin Cancer Res. 2020; 26:290-300.
Morel K L, Sheahan A V, Burkhart D L, Baca S C, Boufaied N, Liu Y, et al. EZH2 inhibition activates a dsRNA-STING-interferon stress axis that potentiates response to PD-1 checkpoint blockade in prostate cancer. Nat Cancer. 2021; 2:444-56.
Zhao E, Maj T, Kryczek I, Li W, Wu K, Zhao L, et al. Cancer mediates effector T cell dysfunction by targeting microRNAs and EZH2 via glycolysis restriction. Nat Immunol [Internet]. NIH Public Access; 2016 [cited 2018 Mar. 17]; 17:95-103.
Tumes D J, Onodera A, Suzuki A, Shinoda K, Endo Y, Iwamura C, et al. The polycomb protein Ezh2 regulates differentiation and plasticity of CD4(+) T helper type 1 and type 2 cells. Immunity. United States; 2013; 39:819-32.
Tong Q, He S, Xie F, Mochizuki K, Liu Y, Mochizuki I, et al. Ezh2 regulates transcriptional and posttranslational expression of T-bet and promotes Th1 cell responses mediating aplastic anemia in mice. J Immunol. 2014; 192:5012-22.
Drosos Y, Myers J A, Xu B, Mathias K M, Beane E C, Radko-Juettner S, et al. NSD1 mediates antagonism between SWI/SNF and polycomb complexes and is required for transcriptional activation upon EZH2 inhibition. Mol Cell [Internet]. Elsevier; 2022; 82:2472-2489.e8.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

1. A method of treating an individual for a solid tumor comprising cancer cells deficient in in histone methyl transferase NSD1 (nuclear receptor binding SET domain protein 1) activity, the method comprising:
administering an effective dose of an inhibitor of lysine-specific demethylase 2A (KDM2A) to the individual.

2. The method of claim 1, wherein the solid tumor has an immune-cold microenvironment.

3. The method of claim 1, wherein inhibition of KDM2A results in increased expression of one or more chemokines involved in T cell recruitment.

4. The method of claim 3, wherein the one or more chemokines comprise CXCL9 and CXCL10.

5. The method of claim 1, wherein the cancer cells are phenotyped or genotyped for the presence of mutations that decrease NSD1 activity prior to treatment.

6. The method of claim 5, wherein an individual selected for treatment comprises inactivating mutations in one or both NSD1 alleles.

7. The method of claim 1, wherein the cancer is a carcinoma.

8. The method of claim 7, wherein the carcinoma is a squamous cell carcinoma.

9. The method of claim 8, wherein the squamous cell carcinoma is a head and neck squamous cell carcinoma (HNSCC).

10. The method of claim 1, wherein the inhibitor of KDM2A is daminozide.

11. The method of claim 1, wherein administration of a KDM2A inhibitor is combined with administration of an effective dose of an immune checkpoint inhibitor (ICI), or an agent that antagonizes an immune inhibitory molecule.

12. The method of claim 11, wherein the combination comprises an antibody that antagonizes an immune checklpoint protein selected from CTLA4; PD1 and/or PDL1; TIM3; and LAG3.

13. The method of claim 11, wherein the combination comprises an antibody that agonizes an immune costimulatory molecule selected from CD40 and OX40.

14. The method of claim 1, further comprising administering an antibody that binds to an antigen on the targeted tumor cell.

15. The method of claim 1, wherein administration of a KDM2A inhibitor is combined with administration of an adoptive immune cell therapy.

16. The method of claim 15, wherein the adoptive immune cell therapy comprises administering an effective dose of CAR T-cells.

17. A method of treating an individual for a solid tumor, the method comprising:
administering an effective dose of an inhibitor of lysine-specific demethylase 2A (KDM2A) to the individual in combination with one or more of:
administration of an effective dose of an immune checkpoint inhibitor (ICI), administration of an effective dose of an agent that antagonizes an immune inhibitory molecule; and adoptive immune cell therapy.

18. The method of claim 1, wherein the combination provides for a therapeutically effective dose of agents with a clinically significant increase in killing of tumor cells relative to the treatment in the absence of the inhibitor of KDM2A.
